(19) United States
(12) Patent Application Publication
(10) Pub. No.: US 2003/0099992 A1
(43) Pub. Date: May 29, 2003

(54) Genes associated with mast cell activation

(76) Inventors: Karl Nocka, Cambridge, MA (US); Sun Lu, Potomac, MD (US); Quintus Medley, Cambridge, MA (US); Daniel Thomis, Marblehead, MA (US); Jessie Gu, Waltham, MA (US)

Correspondence Address: MORGAN LEWIS & BOCKIUS LLP, 1111 PENNSYLVANIA AVENUE NW, WASHINGTON, DC, 20004, US

(21) Appl. No.: 10/260,715

(22) Filed: Oct. 1, 2002

Related U.S. Application Data
(60) Provisional application No. 60/325,536, filed on Oct. 1, 2001.

(51) Int. Cl. C12Q 1/68; G01N 33/53; G01N 33/567; A01K 67/00; C07H 21/04; C12N 9/64; C07K 14/47; C12P 21/02; C12N 5/06

(52) U.S. Cl. 435/6; 800/8; 435/7.2; 435/69.3; 435/226; 435/320.1; 435/325; 530/350; 536/23.2

(57) ABSTRACT

The invention relates generally to the changes in gene expression in mast cells and tissues removed from patients with allergic hypersensitivity. The invention specifically relates to the genes MC21, MC22, MC25, MC33, MC36, and MC39, which are differentially expressed in mast cells compared to normal tissues and in resting mast cells versus activated mast cells.

### FIELD OF THE INVENTION

The invention relates generally to the changes in gene expression in mast cells and tissues removed from patients with allergic hypersensitivity. The invention specifically relates to the genes MC21, MC22, MC25, MC33, MC36 and MC39, which are differentially expressed in mast cells compared to normal tissues and in resting mast cells versus activated mast cells.

### BACKGROUND OF THE INVENTION

Allergic Hypersensitivity

The inflammatory response characteristic of allergic or hypersensitivity reactions can be elicited by extrinsic antigens such as pollen, dust, food, and chemicals in the environment. There are four main classes of hypersensitivity reactions, which are distinguished by the type of immune cells and antibodies involved and the pathologies produced. In the most common IgE-dependent allergic reactions, the inflammatory response involves mast cell degranulation, or emptying of the granules, triggered by allergen interaction with IgE molecules on the mast cell surface. Present in large numbers in epithelial tissue, mast cells have high-affinity IgE receptors on their surface.

Inhaled allergens initiate respiratory allergies such as allergic rhinitis, hay fever and asthma, while ingested allergens may cause food allergies. Injected allergens, such as antibiotics and insect venoms, may cause life-threatening anaphylactic reactions.

The cytoplasmic granules of the mature mast cell contain mediators of allergies, such as histamine, heparin, and proteases. Newly formed lipid mediators, such as leukotrienes and prostaglandins are also rapidly synthesized and released from mast cells upon activation. Small quantities of these mediators released on the mucosal surfaces of the respiratory system cause the symptoms associated with allergic rhinitis for example itchy watery eyes, runny nose, and sneezing. Many of the symptoms associated with asthma are directly associated with the effects of mediators released from the mast cell as well as indirectly through other cells that are recruited to the lung by these mediators. Larger amounts of these mediators contract the respiratory smooth muscles, limit breathing, and may cause an anaphylactic reaction which can be fatal. Release of these mediators in the skin causes edema, erythema, and wheal formation, i.e., urticaria.

Urticaria is a skin condition characterized by the appearance of intensely itching wheals or welts with elevated centers and a surrounding area of erythema (redness). Wheals are usually distributed over the trunk and extremities of the body, but they may occur on any epithelial or mucosal surface. Urticaria may be acute, lasting six weeks or less, or chronic. A related skin condition, angiodema, with similar, but non-pruritic, sores, affects deeper levels of skin tissues. It is estimated that 10-20% of the population, usually children, suffers from urticaria or angiodema or both simultaneously at one time or another (Frank, M. M., Cecil Textbook of Medicine, 20^{th} Ed., Bennet and Plum eds., chap. 19, pp. 1408-1412, W. B. Saunders Co., Philadelphia, 1996). The most common and most effective treatment to date is administration of antihistamines or glucocorticoids, and in some cases epinephrine. Where antihistamines prove ineffective, the symptoms persist for years, or even decades. In about 70% of urticaria/angiodema cases, the cause of the disease is not found. Typical sources, however, are ingested allergens from foods or drugs, such as non-steroidal anti-inflammatories, or autoimmune-type allergens, such as antithyroid, anti-IgG or anti-IgE autoantibodies. Other causes include physical factors, e.g., heat, cold, pressure and sun.

Treatment of Allergies and Asthma

Conventional therapeutic compounds, such as antihistamines (H1 antagonists), prevent histamine from binding to H1 receptors. Antihistamines may be taken to block the effect of histamine released from mast cell granules, but they have no effect on the activities of the other co-released vasoactive compounds. Cromolyn sodium and nedocromil are effective in some patients and may block mast cell degranulation and, therefore, the release of histamine as well as other mediators from mast cells. Lipoxygenase inhibitors or leukotriene antagonists may specifically block the effects of leukotrienes released from mast cells. Other agents such as glucocorticoids, theophylline, and beta-agonists play important roles in the control of asthma. They have all been shown to have an inhibitory effect on either mast cell development or activation. A new approach of targeting IgE directly with monoclonal-antibody therapy has been found to be effective in some patients with rhinitis as well as asthma and has demonstrated the importance of this trigger on the mast cell in these diseases.

Cellular and Molecular Events Involved in Mast Cell Function and Activation

It has been demonstrated that within activated mast cells, the expression levels of a number of genes are changed compared to unactivated cells. Generally, however, little is known about the cellular and molecular events associated with mast cell degranulation and regranulation of mast cells, or about the events and stages of cellular maturation that lead to a functionally mature mast cell.

While the changes in the expression levels of a number of individual genes have been identified, the investigation of the global changes in gene expression has not been reported in human mast cells. Accordingly, there exists a need for the investigation of the changes in gene expression levels as well as the need for the identification of new molecular markers associated with mast cell maturation, activation, degranulation and regranulation. Furthermore, if intervention is expected to be successful preventing or reducing allergic hypersensitivity, means of accurately assessing mast cell maturation and the early stages of activation need to be established. One way to accurately assess the early cellular events involved in mast cell activation is to identify markers which are uniquely associated with the process. Likewise, the development of therapeutics to prevent or stop IgE-mediated allergic reactions relies on the identification of genes responsible for mast cell maturation, activation and de- and regranulation.

### SUMMARY OF THE INVENTION

The present invention is based on the discovery of new gene families that are differentially expressed in mast cells that have been activated through the IgE receptor. These exhibit a high level of expression in resting mast cells as compared to non-mast cell populations or whole tissues.

The invention includes isolated nucleic acid molecules selected from the group consisting of an isolated nucleic acid molecule that encodes the amino acid sequence of SEQ ID NO: 2, 4, 6, 8, 10, 12, 14, or 16; an isolated nucleic acid molecule that encodes a fragment of at least 6 amino acids of SEQ ID NO: 2, 4, 6, 8, 10, 12, 14, or 16; an isolated nucleic acid molecule which hybridizes to the complement of a nucleic acid molecule comprising SEQ ID NO: 1, 3, 5, 7, 9, 11, 13, or 15; and an isolated nucleic acid molecule which hybridizes to the complement of a nucleic acid molecule that encodes the amino acid sequence of SEQ ID NO: 2, 4, 6, 8, 10, 12, 14, or 16. Nucleic acid molecules of the invention may encode a protein having at least about 35%, 40%, 50%, 60%, or 65% amino acid sequence identity to SEQ ID NO: 2, 4, 6, 8, 10, 12, 14, or 16, preferably at least about 70% or 75% sequence identity, more preferably at least about 80-85% sequence identity, even more preferably at least about 90% and most preferably 95% sequence identity to SEQ ID NO: 2, 4, 6, 8, 10, 12, 14, or 16.

The present invention further includes the nucleic acid molecules operably linked to one or more expression control elements, including vectors comprising the isolated nucleic acid molecules. The invention further includes host cells transformed to contain the nucleic acid molecules of the invention and methods for producing a protein comprising culturing a host cell transformed with a nucleic acid molecule of the invention under conditions in which the protein is expressed.

The invention further provides an isolated polypeptide selected from the group consisting of an isolated polypeptide comprising the amino acid sequence of SEQ ID NO: 2, 4, 6, 8, 10, 12, 14, or 16, an isolated polypeptide comprising a functional or antigenic fragment of at least 6 amino acids of SEQ ID NO: 2, 4, 6, 8, 10, 12, 14, or 16, an isolated polypeptide comprising conservative amino acid substitutions of SEQ ID NO: 24, 6, 8, 10, 12, 14, or 16, and an isolated polypeptide comprising naturally occurring amino acid sequence variants of SEQ ID NO: 2, 4, 6, 8, 10, 12, 14, or 16. Polypeptides of the invention also include polypeptides with an amino acid sequence having at least about 35%, 40%, 50%, 60%, 65%, 70% or 75% amino acid sequence identity with the sequence set forth in SEQ ID NO: 2, 4, 6, 8, 10, 12, 14, or 16; more preferably at least about 80%, even more preferably at least about 90%, and most preferably at least about 95% sequence identity with the full length sequence set forth in SEQ ID NO: 2, 4, 6, 8, 10, 12, 14, or 16.

The invention further provides an isolated antibody or antigen binding fragment that specifically binds to a polypeptide of the invention, including monoclonal and polyclonal antibodies.

The invention further provides methods of identifying an agent which modulates the expression of a nucleic acid encoding a protein of the invention, comprising: exposing cells which express the nucleic acid to the agent; and determining whether the agent modulates expression of said nucleic acid, thereby identifying an agent which modulates the expression of a nucleic acid encoding the protein.

The invention further provides methods of identifying an agent which modulates at least one activity of a protein of the invention, comprising: exposing cells which express the protein to the agent; and determining whether the agent modulates at least one activity of said protein, thereby identifying an agent which modulates at least one activity of the protein.

The invention further provides methods of identifying binding partners for a protein of the invention, comprising: exposing said protein to a potential binding partner; and determining if the potential binding partner binds to said protein, thereby identifying binding partners for the protein.

The present invention further provides methods of modulating the expression of a nucleic acid encoding a protein of the invention, comprising administering an effective amount of an agent which modulates the expression of a nucleic acid encoding the protein. The invention also provides methods of modulating at least one activity of a protein of the invention, comprising administering an effective amount of an agent which modulates at least one activity of the protein.

The present invention further includes non-human transgenic animals modified to contain the nucleic acid molecules of the invention or mutated nucleic acid molecules such that expression of the encoded polypeptides of the invention is prevented.

The invention further provides methods of diagnosing states of IgE-mediated hypersensitivity, urticaria or mastocytosis comprising determining the level of expression of a nucleic acid molecule of the invention or polypeptide of the invention.

### BRIEF DESCRIPTION OF THE DRAWINGS

FIG. 1 shows the relative expression of AF150143 (MC21) using the Affymetrix human GeneChip 95K chip set in samples from cultured human mast cells, isolated human hematopoietic cells, and various normal human tissues.

FIG. 2 shows a Northern blot, in which the expression level of a 1.79 kb mRNA species corresponding to SEQ ID NO: 1 (clone BD9 (MC21)) was measured in human cultured mast cells or tissues. Lane 1) activated mast cells, Lane 2) resting mast cells, Lane 3) kidney, Lane 4) fetal liver, Lane 5) heart, Lane 6) placenta.

FIG. 3 shows a Kile-Doolittle Hydropathy plot of the amino acid sequence of the encoded MC21 protein.

FIG. 4 shows a Parker antigenicity plot of the amino acid sequence of the encoded MC21 protein.

FIG. 5 shows the relative expression of MC22 using the Affymetrix human GeneChip 95K chip set in samples from cultured human mast cells, isolated human hematopoietic cells, and various normal human tissues.

FIG. 6 shows the relative expression of MC25 using the Affymetrix human GeneChip 95K chip set in samples from cultured human mast cells, isolated human hematopoietic cells, and various normal human tissues.

FIG. 7 shows the relative expression of MC33 using the Affymetrix human GeneChip 95K chip set in samples from cultured human mast cells, isolated human hematopoietic cells, and various normal human tissues.

FIG. 8 shows the relative expression of MC36 using the Affymetrix human GeneChip 95K chip set in samples from cultured human mast cells, isolated human hematopoietic cells, and various normal human tissues.

FIG. 9 shows the relative expression of MC39 using the Affymetrix human GeneChip 95K chip set in samples from cultured human mast cells, isolated human hematopoietic cells, and various normal human tissues.

### DETAILED DESCRIPTION OF THE PREFERRED EMBODIMENT

I. General Description

The present invention is based, in part, on the identification of a new gene family that is highly expressed in mast cells, with limited expression in other cell types or tissues. This gene family corresponds to the human cDNA of SEQ ID NO: 1. Genes that encode the human protein of SEQ ID NO: 2 may also be found in other animal species, particularly mammalian species.

MC21 is initially encoded with a signal peptide which spans amino acid residues 1-38 of SEQ ID NO: 2. The mature MC21 protein is a transmembrane protein which spans amino acid residues 39 to 323 of SEQ ID NO: 2. The extracellular domain of mature MC21 comprises a V-set Ig domain which may bind sialic acid, at about amino acid residues 42 to 161 and a C2 immunoglobulin domain at about amino acids 162 to 254 of SEQ ID NO: 2. The transmembrane domain of MC21 is found at about amino acid residues 279 to 301 of SEQ ID NO: 2 and MC21 has a short cytoplasmic tail from about amino acid 302 to 323 of SEQ ID NO: 2. It is notable that MC21 has a lysine residue within the transmembrane domain at amino acid position 290 of SEQ ID NO: 2, a feature common to proteins which are associated within the transmembrane domain with another protein domain. Three cDNAs have been identified which contain part or all of the MC21 open reading frame, BA4, BD9 and OB4. OB4 is extended 5′ versus the other 2 cDNA clones and contains the entire MC21 open reading frame.

The present invention further includes additional new genes that are highly expressed in mast cells, with limited expression in other cell types or tissues. These genes correspond to the human cDNA sequences termed MC22.1 (SEQ ID NO: 3), which encodes the polypeptide of SEQ ID NO: 4; MC22.2 (SEQ ID NO: 5), which encodes the polypeptide of SEQ ID NO: 6; MC25 (SEQ ID NO: 7), which encodes the polypeptide of SEQ ID NO: 8; MC33 (SEQ ID NO: 9), which encodes the polypeptide of SEQ ID NO: 10; MC36 (SEQ ID NO: 11), which encodes the polypeptide of SEQ ID NO: 12; MC39 (first open reading frame (ORF); SEQ ID NO: 13), which encodes the polypeptide of SEQ ID NO: 14; and MC39 (second open reading frame (ORF); SEQ ID NO: 15), which encodes the polypeptide of SEQ ID NO: 16. Genes that encode the human proteins supra may also be found in other animal species, particularly mammalian species.

MC22, which has two splice variants termed here MC22.1 (SEQ ID NO: 3) and MC22.2 (SEQ ID NO: 5), is a human ortholog of the rat protein tomosyn with 89% identity. Tomosyn is a syntaxin- 1-binding protein that forms a complex in the neurotransmitter release process (Fujita, Y. et al. (1998) Neuron 20(5):905-915).

MC21, MC22, MC25, MC33, MC36 and MC39 may be used individually or in combination of at least two members of the group as markers to detect, diagnose or identify an allergic response in a patient. MC21, MC22, MC25, MC33, MC36 and MC39 may also be used individually or in combination of at least two members of the group as markers to detect, determine or identify the state of mast cell activation, i.e., to determine whether a mast cell is in an activated or resting state. The proteins can also serve as targets for agents that modulate gene or protein expression or activity. For example, agents may be identified that modulate biological processes associated with mast cell function, including mast cell degranulation that leads to urticaria.

II. Specific Embodiments

A. The Proteins Associated with Mast Cell Regranulation and/or Allergic Hypersensitivity

The present invention includes isolated proteins, allelic variants of the proteins, and conservative amino acid substitutions of the proteins. As used herein, the “protein” or “polypeptide” refers, in part, to MC21, a protein that has the human amino acid sequence depicted in SEQ ID NO: 2 or fragments thereof; to MC22, which includes the splice variants MC22.1 (a protein that has the human amino acid sequence depicted in SEQ ID NO: 4 or fragments thereof) and MC22.2 (a protein that has the human amino acid sequence depicted in SEQ ID NO: 6 or fragments thereof); to MC25, a protein that has the human amino acid sequence depicted in SEQ ID NO: 8 or fragments thereof; to MC33, a protein that has the human amino acid sequence depicted in SEQ ID NO: 10 or fragments thereof; to MC36, a protein that has the human amino acid sequence depicted in SEQ ID NO: 12 or fragments thereof; or to MC39, including the first open reading frame (a protein that has the human amino acid sequence depicted in SEQ ID NO: 14 or fragments thereof) and the second open reading frame (a protein that has the human amino acid sequence depicted in SEQ ID NO: 16 or fragments thereof). The terms also refer to naturally occurring allelic variants and proteins that have a slightly different amino acid sequence than that specifically recited above. Allelic variants, though possessing a slightly different amino acid sequence than those recited above, will still have the same or similar biological functions associated with these proteins.

As used herein, the family of proteins related to the human amino acid sequence of SEQ ID NO: 2, 4, 6, 8, 10, 12, 14, or 16 refers in part, to proteins that have been isolated from organisms in addition to humans. The methods used to identify and isolate other members of the family of proteins related to these proteins are described below.

The proteins of the present invention are preferably in isolated form. As used herein, a protein is said to be isolated when physical, mechanical or chemical methods are employed to remove the protein from cellular constituents that are normally associated with the protein. A skilled artisan can readily employ standard purification methods to obtain an isolated protein.

The proteins of the present invention further include insertion, deletion, conservative amino acid substitution or splice variants of SEQ ID NO: 2, 4, 6, 8, 10, 12, 14, or 16. As used herein, a conservative variant refers to alterations in the amino acid sequence that do not adversely affect the biological functions of the protein. A substitution, insertion or deletion is said to adversely affect the protein when the altered sequence prevents or disrupts a biological function associated with the protein. For example, the overall charge, structure or hydrophobic/hydrophilic properties of the protein can be altered without adversely affecting a biological activity. Accordingly, the amino acid sequence can be altered, for example to render the peptide more hydrophobic or hydrophilic, without adversely affecting the biological activities of the protein.

Ordinarily, the allelic variants, the conservative substitution variants, and the members of the protein family, will have an amino acid sequence having at least about 35%, 40%, 50%, 60%, 65%, 70% or 75% amino acid sequence identity with the full length sequence set forth in SEQ ID NO: 2, 4, 6, 8, 10, 12, 14, or 16, more preferably at least about 80%, even more preferably at least about 90%, and most preferably at least about 95%, 97% or 99% sequence identity. Identity or homology with respect to such sequences is defined herein as the percentage of amino acid residues in the candidate sequence that are identical with the known peptides, after aligning the sequences and introducing gaps, if necessary, to achieve the maximum percent homology, and not considering any conservative substitutions as part of the sequence identity (see section B for the relevant parameters). Fusion proteins, or N-terminal, C-terminal or internal extensions, deletions, or insertions into the peptide sequence shall not be construed as affecting homology.

Thus, the proteins of the present invention include molecules having the amino acid sequence disclosed in SEQ ID NO: 2, 4, 6, 8, 10, 12, 14, or 16; fragments thereof preferably having a consecutive sequence of SEQ ID NO: 2, 4, 6, 8, 10, 12, 14, or 16 of at least about 6 or 10, 15 or 20, or 25 or 30 amino acid residues, more preferably 35 or 40 amino acid residues, even more preferably 45 or 50 amino acid residues, yet more preferably 55 or 60, still more preferably 65 or 70 amino acid residues and most preferably at least 75 or more amino acid residues; amino acid sequence variants wherein one or more amino acid residues has been inserted N- or C-terminal to, or within, the disclosed coding sequence; and amino acid sequence variants of the disclosed sequence, or their fragments as defined above, that have been substituted by at least one residue. Such fragments, also referred to as peptides or polypeptides, may contain antigenic regions, functional regions of the protein identified as regions of the amino acid sequence which correspond to known protein domains, as well as regions of pronounced hydrophilicity. The regions are all easily identifiable by using commonly available protein sequence analysis software such as MacVector (Oxford Molecular).

Proteins of the invention further include the mature MC21 protein without the signal sequence. For example, the invention includes a polypeptide consisting of or comprising about amino acid residues 39-323 of SEQ ID NO: 2. Fragments of the MC21 protein encompassed by the present invention also include the extracellular domain attached to the transmembrane domain, but without the signal peptide or the cytoplasmic tail. For example, the invention includes a polypeptide consisting of or comprising about amino acid residues 39 to 301 of SEQ ID NO: 2. The invention also includes a soluble form of MC21 consisting essentially of the extracellular domain of the protein. For example, the invention includes a polypeptide consisting of or comprising about amino acid residues 39 to 278 of SEQ ID NO: 2. The invention further includes variant, altered or mutant forms of the mature MC21 protein and of the fragments thereof which have amino acid deletions, additions or substitutions to the amino acid sequence of SEQ ID NO: 2. Said amino acid deletions, additions or substitutions may be silent, not changing the biological and/or immunological function or properties of MC21, such as a conservative substitution. Said amino acid deletions, additions or substitutions may also destroy or ablate the biological and/or immunological function or properties of MC21. Further, said amino acid deletions, additions or substitutions may enhance or diminish the biological and/or immunological function or properties of MC21.

Proteins of the invention further include the MC22.1 polypeptide. For example, the invention includes a polypeptide consisting of or comprising about amino acid residues 1-1151 of SEQ ID NO: 4. The invention further includes variant, altered or mutant forms of the MC22.1 protein and of the fragments thereof which have amino acid deletions, additions or substitutions to the amino acid sequence of SEQ ID NO: 4. Said amino acid deletions, additions or substitutions may be silent, not changing the biological and/or immunological function or properties of MC22.1, such as a conservative substitution. Said amino acid deletions, additions or substitutions may also destroy or ablate the biological and/or immunological function or properties of MC22.1. Further, said amino acid deletions, additions or substitutions may enhance or diminish the biological and/or immunological function or properties of MC22.1.

Proteins of the invention further include the MC22.2 polypeptide. For example, the invention includes a polypeptide consisting of or comprising about amino acid residues 1- 1115 of SEQ ID NO: 6. The invention further includes variant, altered or mutant forms of the MC22.2 protein and of the fragments thereof which have amino acid deletions, additions or substitutions to the amino acid sequence of SEQ ID NO: 6. Said amino acid deletions, additions or substitutions may be silent, not changing the biological and/or immunological function or properties of MC22.2, such as a conservative substitution. Said amino acid deletions, additions or substitutions may also destroy or ablate the biological and/or immunological function or properties of MC22.2. Further, said amino acid deletions, additions or substitutions may enhance or diminish the biological and/or immunological function or properties of MC22.2.

Proteins of the invention further include the MC25 protein. For example, the invention includes a polypeptide consisting of or comprising about amino acid residues 1-1259 of SEQ ID NO: 8. The invention further includes variant, altered or mutant forms of the MC25 protein and of the fragments thereof which have amino acid deletions, additions or substitutions to the amino acid sequence of SEQ ID NO: 8. Said amino acid deletions, additions or substitutions may be silent, not changing the biological and/or immunological function or properties of MC25, such as a conservative substitution. Said amino acid deletions, additions or substitutions may also destroy or ablate the biological and/or immunological function or properties of MC25. Further, said amino acid deletions, additions or substitutions may enhance or diminish the biological and/or immunological function or properties of MC25.

Proteins of the invention further include the MC33 protein. For example, the invention includes a polypeptide consisting of or comprising about amino acid residues 1-350 of SEQ ID NO: 10. The invention further includes variant, altered or mutant forms of the MC33 protein and of the fragments thereof which have amino acid deletions, additions or substitutions to the amino acid sequence of SEQ ID NO: 10. Said amino acid deletions, additions or substitutions may be silent, not changing the biological and/or immunological function or properties of MC33, such as a conservative substitution. Said amino acid deletions, additions or substitutions may also destroy or ablate the biological and/or immunological function or properties of MC33. Further, said amino acid deletions, additions or substitutions may enhance or diminish the biological and/or immunological function or properties of MC33.

Proteins of the invention further include the MC36 protein. For example, the invention includes a polypeptide consisting of or comprising about amino acid residues 1-398 of SEQ ID NO: 12. The invention further includes variant, altered or mutant forms of the MC36 protein and of the fragments thereof which have amino acid deletions, additions or substitutions to the amino acid sequence of SEQ ID NO: 12. Said amino acid deletions, additions or substitutions may be silent, not changing the biological and/or immunological function or properties of MC36, such as a conservative substitution. Said amino acid deletions, additions or substitutions may also destroy or ablate the biological and/or immunological function or properties of MC36. Further, said amino acid deletions, additions or substitutions may enhance or diminish the biological and/or immunological function or properties of MC36.

Proteins of the invention further include the polypeptide encoded by the MC39 first ORF polypeptide. For example, the invention includes a polypeptide consisting of or comprising about amino acid residues 1-107 of SEQ ID NO: 14. The invention further includes variant, altered or mutant forms of the MC39 first ORF polypeptide and of the fragments thereof which have amino acid deletions, additions or substitutions to the amino acid sequence of SEQ ID NO: 14. Said amino acid deletions, additions or substitutions may be silent, not changing the biological and/or immunological function or properties of the MC39 first ORF polypeptide, such as a conservative substitution. Said amino acid deletions, additions or substitutions may also destroy or ablate the biological and/or immunological function or properties of the MC39 first ORF polypeptide. Further, said amino acid deletions, additions or substitutions may enhance or diminish the biological and/or immunological function or properties of the MC39 first ORF polypeptide.

Proteins of the invention further include the polypeptide encoded by the MC39 second ORF polypeptide. For example, the invention includes a polypeptide consisting of or comprising about amino acid residues 1-83 of SEQ ID NO: 16. The invention further includes variant, altered or mutant forms of the MC39 second ORF polypeptide and of the fragments thereof which have amino acid deletions, additions or substitutions to the amino acid sequence of SEQ ID NO: 16. Said amino acid deletions, additions or substitutions may be silent, not changing the biological and/or immunological function or properties of the MC39 second ORF polypeptide, such as a conservative substitution. Said amino acid deletions, additions or substitutions may also destroy or ablate the biological and/or immunological function or properties of the MC39 second ORF polypeptide. Further, said amino acid deletions, additions or substitutions may enhance or diminish the biological and/or immunological function or properties of the MC39 second ORF polypeptide.

Contemplated variants further include those containing predetermined mutations by, e.g., homologous recombination, site-directed or PCR mutagenesis, and the corresponding proteins of other animal species, including but not limited to rabbit, mouse, rat, porcine, bovine, ovine, equine and non-human primate species, and the alleles or other naturally occurring variants of the family of proteins; and derivatives wherein the protein has been covalently modified by substitution, chemical, enzymatic, or other appropriate means with a moiety other than a naturally occurring amino acid (for example a detectable moiety such as an enzyme or radioisotope).

As described below, members of the family of proteins can be used: (1) as a diagnostic marker; (2) to identify agents which modulate at least one activity of the protein; (3) to identify binding partners for the protein, (4) as an antigen to raise polyclonal or monoclonal antibodies, and (5) as a therapeutic agent or target.

B. Nucleic Acid Molecules

The present invention further includes nucleic acid molecules that encode the protein having SEQ ID NO: 2, 4, 6, 8, 10, 12, 14, or 16 and the related proteins herein described, preferably in isolated form. As used herein, “nucleic acid” is defined as RNA or DNA or related molecules that encodes a protein or peptide as defined above, is complementary to a nucleic acid sequence encoding such peptides, hybridizes to such a nucleic acid and remains stably bound to it under appropriate stringency conditions, or encodes a polypeptide sharing at least about 35%, 40%, 50%, 60%, 65%, 70% or 75% sequence identity, preferably at least about 80%, more preferably at least about 85%, and even more preferably at least about 90%, 95%, 97% or 99% or more identity with the full-length peptide sequence of SEQ ID NO: 2, 4, 6, 8, 10, 12, 14, or 16. The “nucleic acid molecules” of the invention further include nucleic acid molecules that share at least about 70% or 75% sequence identity, preferably at least about 80%, more preferably at least about 85%, and even more preferably at least about 90% and most preferably 95%, 97%, 99% or more identity with the nucleotide sequence of SEQ ID NO: 1, 3, 5, 7, 9, 11, 13, or 15 or the full length of the open reading frame defined therein. Specifically contemplated are genomic DNA, cDNA, mRNA and antisense molecules, as well as nucleic acids based on alternative backbones or including alternative bases whether derived from natural sources or synthesized. Such nucleic acids, however, are defined further as being novel and unobvious over any prior art nucleic acid including that which encodes, hybridizes under appropriate stringency conditions, or is complementary to nucleic acid encoding a protein according to the present invention.

Homology or identity at the nucleotide or amino acid sequence level is determined by BLAST (Basic Local Alignment Search Tool) analysis using the algorithm employed by the programs blastp, blastn, blastx, tblastn and tblastx (Altschul, S. F. et al., Nucleic Acids Res 25: 3389-3402 (1997) and Karlin et al., Proc Natl Acad Sci USA 87:2264-2268 (1990), both fully incorporated by reference) which are tailored for sequence similarity searching. The approach used by the BLAST program is to first consider similar segments, with and without gaps, between a query sequence and a database sequence, then to evaluate the statistical significance of all matches that are identified and finally to summarize only those matches which satisfy a pre-selected threshold of significance. For a discussion of basic issues in similarity searching of sequence databases, see Altschul et al., (1994) (Nature Genetics 6, 119-129) which is fully incorporated by reference. The search parameters for histogram, descriptions, alignments, expect (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix (Henikoffet al., (1992) Proc. Natl. Acad. Sci. USA 89, 10915-10919, fully incorporated by reference), recommended for query sequences over 85 in length (nucleotide bases or amino acids).

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Gap comparison between sequences, available in the Accelrys' Wisconsin Package version 10.2, uses DNA parameters GAP=50 (gap creation penalty) and LEN=3 (gap extension penalty) and the equivalent settings in protein comparisons are GAP=8 and LEN=2.

“Stringent conditions” include those that (1) employ low ionic strength and high temperature for washing, for example, 0.015 M NaCl/0.0015 M sodium citrate/0.1% SDS at 50 C, or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42 C. Another example is hybridization in 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS, and 10% dextran sulfate at 42 C, with washes at 42 C in 0.2×SSC and 0.1% SDS. A skilled artisan can readily determine and vary the stringency conditions appropriately to obtain a clear and detectable hybridization signal. Preferred molecules are those that hybridize under the above conditions to the complement of SEQ ID NO: 1, 3, 5, 7, 9, 11, 13, or 15 and which encode a functional protein. Even more preferred hybridizing molecules are those that hybridize under the above conditions to the complement strand of the open reading frame of SEQ ID NO: 1, 3, 5, 7,9, 11, 13, or 15.

As used herein, a nucleic acid molecule is said to be “isolated” when the nucleic acid molecule is substantially separated from contaminant nucleic acid molecules encoding other polypeptides.

The present invention further provides fragments of the nucleic acid molecules. As used herein, a fragment of an encoding nucleic acid molecule refers to a small portion of the entire protein coding sequence. The size of the fragment will be determined by the intended use. For example, if the fragment is chosen so as to encode an active portion of the protein, the fragment will need to be large enough to encode the functional region(s) of the protein. For instance, fragments which encode peptides corresponding to predicted antigenic regions may be prepared. If the fragment is to be used as a nucleic acid probe or PCR primer, then the fragment length is chosen so as to obtain a relatively small number of false positives during probing/priming (see the discussion in Section H).

Fragments of the nucleic acid molecules of the present invention (i.e., synthetic oligonucleotides) that are used as probes or specific primers for the polymerase chain reaction (PCR), or to synthesize gene sequences encoding proteins of the invention, can easily be synthesized by chemical techniques, for example, the phosphotriester method of Matteucci et al., (1981) (J. Am. Chem. Soc. 103, 3185-3191) or using automated synthesis methods. In addition, larger DNA segments can readily be prepared by well-known methods, such as synthesis of a group of oligonucleotides that define various modular segments of the gene, followed by ligation of oligonucleotides to build the complete modified gene.

The nucleic acid molecules of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can readily employ any such label to obtain labeled variants of the nucleic acid molecules of the invention.

Modifications to the primary structure of the nucleic acid molecules by deletion, addition, or alteration of the nucleotide sequence can be made without destroying the activity of the encoded proteins. Such substitutions or other alterations result in proteins having an amino acid sequence falling within the contemplated scope of the present invention.

C. Isolation of other Related Nucleic Acid Molecules

As described above, the identification and characterization of the nucleic acid molecule having SEQ ID NO: 1, 3, 5, 7, 9, 11, 13, or 15 allows a skilled artisan to isolate nucleic acid molecules that encode other members of the protein family in addition to the sequences herein described.

For instance, a skilled artisan can readily use the amino acid sequence of SEQ ID NO: 2, 4, 6, 8, 10, 12, 14 or 16 to generate antibody probes to screen expression libraries prepared from appropriate cells. Typically, polyclonal antiserum from mammals such as rabbits immunized with the purified protein (as described below) or monoclonal antibodies can be used to probe a mammalian cDNA or genomic expression library, such as lambda gt11 library, to obtain the appropriate coding sequence for other members of the protein family. The cloned cDNA sequence can be expressed as a fusion protein, expressed directly using its own control sequences, or expressed by constructions using control sequences appropriate to the particular host used for expression of the enzyme.

Alternatively, a portion of the coding sequence herein described can be synthesized and used as a probe to retrieve DNA encoding a member of the protein family from any mammalian organism. Oligomers containing approximately 18-20 nucleotides (encoding about a 6-7 amino acid stretch) are prepared and used to screen genomic DNA or cDNA libraries to obtain hybridization under stringent conditions or conditions of sufficient stringency to eliminate an undue level of false positives.

Additionally, pairs of oligonucleotide primers can be prepared for use in a polymerase chain reaction (PCR) to selectively clone an encoding nucleic acid molecule. A PCR denature/anneal/extend cycle for using such PCR primers is well known in the art and can readily be adapted for use in isolating other encoding nucleic acid molecules.

Nucleic acid molecules encoding other members of the protein family may also be identified in existing genomic or other sequence information using any available computational method, including but not limited to: PSI-BLAST (Altschul, et al. (1997) Nucleic Acids Res. 25:3389-3402); PHI-BLAST (Zhang, et al. (1998), Nucleic Acids Res. 26:3986-3990), 3D-PSSM (Kelly et al. (2000) J. Mol. Biol. 299(2): 499-520); and other computational analysis methods (Shi et al. (1999) Biochem. Biophys. Res. Commun. 262(1):132-8 and Matsunami et. al (2000) Nature 404(6778):601-4.

D. rDNA Molecules Containing a Nucleic Acid Molecule

The present invention further includes recombinant DNA molecules (rDNAs) that contain a coding sequence. As used herein, a rDNA molecule is a DNA molecule that has been subjected to molecular manipulation in situ. Methods for generating rDNA molecules are well known in the art, for example, see Sambrook et al., (1989) Molecular Cloning—A Laboratory Manual, Cold Spring Harbor Laboratory Press. In the preferred rDNA molecules, a coding DNA sequence is operably linked to expression control sequences and/or vector sequences.

The choice of vector and/or expression control sequences to which one of the protein family encoding sequences of the present invention is operably linked depends directly, as is well known in the art, on the functional properties desired, e.g., protein expression, and the host cell to be transformed. A vector contemplated by the present invention is at least capable of directing the replication or insertion into the host chromosome, and preferably also expression, of the structural gene included in the rDNA molecule.

Expression control elements that are used for regulating the expression of an operably linked protein encoding sequence are known in the art and include, but are not limited to, inducible promoters, constitutive promoters, secretion signals, and other regulatory elements. Preferably, the inducible promoter is readily controlled, such as being responsive to a nutrient in the host cell's medium.

In one embodiment, the vector containing a coding nucleic acid molecule will include a prokaryotic replicon, i.e., a DNA sequence having the ability to direct autonomous replication and maintenance of the recombinant DNA molecule extrachromosomally in a prokaryotic host cell, such as a bacterial host cell, transformed therewith. Such replicons are well known in the art. In addition, vectors that include a prokaryotic replicon may also include a gene whose expression confers a detectable marker such as a drug resistance. Typical bacterial drug resistance genes are those that confer resistance to ampicillin or tetracycline.

Vectors that include a prokaryotic replicon can further include a prokaryotic or bacteriophage promoter capable of directing the expression (transcription and translation) of the coding gene sequences in a bacterial host cell, such as E. coli. A promoter is an expression control element formed by a DNA sequence that permits binding of RNA polymerase and transcription to occur. Promoter sequences compatible with bacterial hosts are typically provided in plasmid vectors containing convenient restriction sites for insertion of a DNA segment of the present invention. Typical of such vector plasmids are pUC8, pUC9, pBR322 and pBR329 available from BioRad Laboratories, (Richmond, Calif.), pPL and pKK223 available from Pharmacia (Piscataway, N.J.).

Expression vectors compatible with eukaryotic cells, preferably those compatible with vertebrate cells, can also be used to form rDNA molecules that contain a coding sequence. Eukaryotic cell expression vectors, including viral vectors, are well known in the art and are available from several commercial sources. Typically, such vectors are provided containing convenient restriction sites for insertion of the desired DNA segment. Typical of such vectors are pSVL and pKSV-10 (Pharmacia), pBPV-1/pML2d (International Biotechnologies, Inc.), pTDT1 (ATCC, #31255), the vector pCDM8 described herein, and the like eukaryotic expression vectors.

Eukaryotic cell expression vectors used to construct the rDNA molecules of the present invention may further include a selectable marker that is effective in an eukaryotic cell, preferably a drug resistance selection marker. A preferred drug resistance marker is the gene whose expression results in neomycin resistance, i.e., the neomycin phosphotransferase (neo) gene. (Southern et al., (1982) J. Mol. Anal. Genet. 1, 327-341) Alternatively, the selectable marker can be present on a separate plasmid, and the two vectors are introduced by co-transfection of the host cell, and selected by culturing in the appropriate drug for the selectable marker.

E. Host Cells Containing an Exogenously Supplied Coding Nucleic Acid Molecule

The present invention further includes host cells transformed with a nucleic acid molecule that encodes a protein of the present invention. The host cell can be either prokaryotic or eukaryotic. Eukaryotic cells useful for expression of a protein of the invention are not limited, so long as the cell line is compatible with cell culture methods and compatible with the propagation of the expression vector and expression of the gene product. Preferred eukaryotic host cells include, but are not limited to, yeast, insect and mammalian cells, preferably vertebrate cells such as those from a mouse, rat, monkey or human cell line. Preferred eukaryotic host cells include Chinese hamster ovary (CHO) cells available from the ATCC as CCL61, NIH Swiss mouse embryo cells (NIH/3T3) available from the ATCC as CRL 1658, baby hamster kidney cells (BHK), and the like eukaryotic tissue culture cell lines.

Any prokaryotic host can be used to express a rDNA molecule encoding a protein of the invention. The preferred prokaryotic host is E. coli.

Transformation of appropriate cell hosts with a rDNA molecule of the present invention is accomplished by well-known methods that typically depend on the type of vector used and host system employed. With regard to transformation of prokaryotic host cells, electroporation and salt treatment methods are typically employed, see, for example, Cohen et al., (1972) Proc. Natl. Acad. Sci. USA 69, 2110; and Sambrook et al., (1989) Molecular Cloning—A Laboratory Manual, Cold Spring Harbor Laboratory Press. With regard to transformation of vertebrate cells with vectors containing rDNAs, electroporation, cationic lipid or salt treatment methods are typically employed, see, for example, Graham et al., (1973) Virol. 52, 456; Wigler et al., (1979) Proc. Natl. Acad. Sci. USA 76, 1373-1376.

Successfully transformed cells, i.e., cells that contain a rDNA molecule of the present invention, can be identified by well known techniques including the selection for a selectable marker. For example, cells resulting from the introduction of an rDNA of the present invention can be cloned to produce single colonies. Cells from those colonies can be harvested, lysed and their DNA content examined for the presence of the rDNA using a method such as that described by Southern, J. Mol. Biol. 98:503, 1975, or Berent et al., (1985) Biotech. 3, 208 or the proteins produced from the cell assayed via an immunological method.

F. Production of Recombinant Proteins Using a rDNA Molecule

The present invention further includes methods for producing a protein of the invention using nucleic acid molecules herein described. In general terms, the production of a recombinant form of a protein typically involves the following steps:

A nucleic acid molecule is first obtained that encodes a protein of the invention, such as a nucleic acid molecule comprising, consisting essentially of or consisting of SEQ ID NO: 1, or the open reading frame defined by nucleotides 70-1038 (or 1041) of SEQ ID NO: 1; a nucleic acid molecule comprising, consisting essentially of or consisting of SEQ ID NO: 3, or the open reading frame defined by nucleotides 1-3453 (or 3456) of SEQ ID NO: 3; a nucleic acid molecule comprising, consisting essentially of or consisting of SEQ ID NO: 5, or the open reading frame defined by nucleotides 1-3345 (or 3348) of SEQ ID NO: 5; a nucleic acid molecule comprising, consisting essentially of or consisting of SEQ ID NO: 7, or the open reading frame defined by nucleotides 317-4093 (or 4096) of SEQ ID NO: 7; a nucleic acid molecule comprising, consisting essentially of or consisting of SEQ ID NO: 9, or the open reading frame defined by nucleotides 1-1050 (or 1053) of SEQ ID NO: 9; a nucleic acid molecule comprising, consisting essentially of or consisting of SEQ ID NO: 11, or the open reading frame defined by nucleotides 1-1194 (or 1197) of SEQ ID NO: 11; a nucleic acid molecule comprising, consisting essentially of or consisting of SEQ ID NO: 13, or the open reading frame defined by nucleotides 1-321 (or 324) of SEQ ID NO: 13; or a nucleic acid molecule comprising, consisting essentially of or consisting of SEQ ID NO: 15, or the open reading frame defined by nucleotides 9-257 (or 260) of SEQ ID NO: 15. If the encoding sequence is uninterrupted by introns, as are these open reading frame, it is directly suitable for expression in any host.

The nucleic acid molecule is then preferably placed in operable linkage with suitable control sequences, as described above, to form an expression unit containing the protein open reading frame. The expression unit is used to transform a suitable host and the transformed host is cultured under conditions that allow the production of the recombinant protein. Optionally the recombinant protein is isolated from the medium or from the cells; recovery and purification of the protein may not be necessary in some instances where some impurities may be tolerated.

Each of the foregoing steps can be done in a variety of ways. For example, the desired coding sequences may be obtained from genomic fragments and used directly in appropriate hosts. The construction of expression vectors that are operable in a variety of hosts is accomplished using appropriate replicons and control sequences, as set forth above. The control sequences, expression vectors, and transformation methods are dependent on the type of host cell used to express the gene and were discussed in detail earlier. Suitable restriction sites can, if not normally available, be added to the ends of the coding sequence so as to provide an excisable gene to insert into these vectors. A skilled artisan can readily adapt any host/expression system known in the art for use with the nucleic acid molecules of the invention to produce recombinant protein.

G. Methods to Identify Binding Partners

Another embodiment of the present invention includes methods of isolating and identifying binding partners of proteins of the invention. By recitation of “binding partner,” the present invention includes ligands or receptors which bind specifically to MC21, MC22.1, MC22.2, MC25, MC33, MC36 or MC39. Further, binding partners also include protein domains which associate via at least one Lys residue within the transmembrane domain at position 282, 292, 293, 294, 296, 298, or 301 of SEQ ID NO: 2.

In general, a protein of the invention is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the protein of the invention can then be removed and further analyzed.

To identify and isolate a binding partner, the entire protein, for instance a protein comprising the entire amino acid sequence of SEQ ID NO: 2 or comprising the sequence of the mature MC21 protein without the signal peptide can be used. For example, a protein or polypeptide consisting of or comprising about amino acid residues 39 to 323 of SEQ ID NO: 2, can be used. Further, a protein or polypeptide consisting of or comprising about amino acid residues 1 to 1151 of SEQ ID NO: 4, 1 to 1115 of SEQ ID NO: 6, 1 to 1259 of SEQ ID NO: 8, 1 to 350 of SEQ ID NO: 10, 1 to 398 of SEQ ID NO: 12, 1 to 107 of SEQ ID NO: 14, or 1 to 83 of SEQ ID NO: 4 can also be used. Alternatively, a fragment of the protein preferably having a consecutive sequence of at least about 25 or 30 amino acid residues, more preferably at least about 35 or 40 amino acid residues, even more preferably at least about 45 or 50 amino acid residues, yet more preferably at least about 55 or 60, still more preferably at least about 65 or 70 amino acid residues and most preferably at least about 75 or more amino acid residues can be used. Further, a fragment of the protein comprising at least the extracellular domain of MC21 or comprising at least the extracellular and transmembrane domains of MC21 can be used to identify and isolate a binding partner. For example, a fragment consisting of or comprising about amino acid residues 39-278 or 39-301 of SEQ ID NO: 2 can be used to identify and isolate a binding partner.

As used herein, a cellular extract refers to a preparation or fraction which is made from a lysed or disrupted cell. The preferred source of cellular extracts will be cells derived from human skin tissue or the human respiratory tract or cells derived from a biopsy sample of human lung tissue in patients with allergic hypersensitivity. Alternatively, cellular extracts may be prepared from normal tissue or available cell lines, particularly granulocytic cell lines.

A variety of methods can be used to obtain an extract of a cell. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, the most preferred being conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the protein of the invention can be immobilized on a solid support. For example, the protein can be attached to a nitrocellulose matrix or acrylic beads. Attachment of the protein to a solid support aids in separating peptide/binding partner pairs from other constituents found in the extract. The identified binding partners can be either a single protein or a complex made up of two or more proteins. Alternatively, binding partners may be identified using a Far-Western assay according to the procedures of Takayama et al., (1997) Methods Mol. Biol. 69:171-184 or Sauder et al., (1996) J. Gen. Virol. 77:991-996 or identified through the use of epitope tagged proteins or GST fusion proteins.

Alternatively, the nucleic acid molecules of the invention can be used in a yeast two-hybrid system. The yeast two-hybrid system has been used to identify other protein partner pairs and can readily be adapted to employ the nucleic acid molecules herein described.

H. Methods to Identify Agents that Modulate the Expression of a Nucleic Acid Encoding the Gene Associated with Mast Cell Degranulation or Allergic Hypersensitivity

Another embodiment of the present invention includes methods for identifying agents that modulate the expression of a nucleic acid encoding a protein of the invention such as a protein having the amino acid sequence of SEQ ID NO: 2, 4, 6, 8, 10, 12, 14, or 16. Such assays may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

In one assay format, cell lines that contain reporter gene fusions between the open reading frame defined by nucleotides 70-1041 of SEQ ID NO: 1, and/or the 5′ and/or 3′ regulatory elements and any assayable fusion partner may be prepared. Alternatively, in the same, a separate or a related assay format, cell lines that contain reporter gene fusions between the open reading frame defined by nucleotides 1-3453 of SEQ ID NO: 3, nucleotides 1-3345 of SEQ ID NO: 5, nucleotides 317-4093 of SEQ ID NO: 7, nucleotides 1-1050 of SEQ ID NO: 9, nucleotides 1-1194 of SEQ ID NO: 11, nucleotides 1-321 of SEQ ID NO: 13, or nucleotides 9-257 of SEQ ID NO: 15, and/or the 5′ and/or 3′ regulatory elements and any assayable fusion partner may be prepared. Numerous assayable fusion partners are known and readily available including the firefly luciferase gene and the gene encoding chloramphenicol acetyltransferase (Alam et al., (1990) Anal. Biochem. 188, 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid of the invention.

Additional assay formats may be used to monitor the ability of the agent to modulate the expression of a nucleic acid encoding a protein of the invention, such as the protein having SEQ ID NO: 2, 4, 6, 8, 10, 12, 14, or 16. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al., (1989) Molecular Cloning—A Laboratory Manual, Cold Spring Harbor Laboratory Press).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., (1989) Molecular Cloning—A Laboratory Manual, Cold Spring Harbor Laboratory Press) or Ausubel et al., (1995) Current Protocols in Molecular Biology, Greene Publishing Co.

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The wafer can then be exposed to total cellular RNA or polyA RNA from a sample under conditions in which the affixed sequences will specifically hybridize. Such solid supports and hybridization methods are widely available, for example, those disclosed by Beattie, (1995) WO 95/11755. By examining for the ability of a given probe to specifically hybridize to an RNA sample from an untreated cell population and from a cell population exposed to the agent, agents which up or down regulate the expression of a nucleic acid encoding the protein having the sequence of SEQ ID NO: 2, 4, 6, 8, 10, 12, 14, or 16 are identified.

Hybridization for qualitative and quantitative analysis of mRNAs may also be carried out by using a RNase Protection Assay (i.e., RPA, see Ma et al. (1996) Methods 10, 273-238). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45 C overnight in a buffer comprising 80% formamide, 40 mM Pipes, pH 6.4, 0.4 M NaCl and 1 mM EDTA. The resulting hybrids are then digested in a buffer comprising 40 g/ml ribonuclease A and 2 g/ml ribonuclease. After deactivation and extraction of extraneous proteins, the samples are loaded onto urea/polyacrylamide gels for analysis.

In another assay format, cells or cell lines are first identified which express the gene products of the invention physiologically. Cell and/or cell lines so identified would be expected to comprise the necessary cellular machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., (1989)).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37 C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

I. Methods to Identify Agents that Modulate the Level of or at Least One Activity of the Protein Associated with Mast Cell Degranulation and/or Allergic Hypersensitivity

Another embodiment of the present invention includes methods for identifying agents that modulate at least one activity of a protein of the invention such as the protein having the amino acid sequence of SEQ ID NO: 2, 4, 6, 8, 10, 12, 14, or 16. Such methods or assays may utilize any means of monitoring or detecting the desired activity.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

Antibody probes are prepared by immunizing suitable mammalian hosts in appropriate immunization protocols using the peptides, polypeptides or proteins of the invention if they are of sufficient length, or, if desired, or if required to enhance immunogenicity, conjugated to suitable carriers. Methods for preparing immunogenic conjugates with carriers such as BSA, KLH, or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein (Nature (1975) 256:495-497) or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonal antibodies or the polyclonal antisera which contain the immunologically significant portion can be used as antagonists, as well as the intact antibodies. Use of immunologically reactive antibody fragments, such as the Fab, Fab′, of F(ab′)₂ fragments is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, such as humanized antibodies. The antibodies may be used in any of the methods described herein, may be used as diagnostic agents or may be used as therapeutic agents.

Agents that are assayed in the above method can be randomly selected or rationally selected or designed. As used herein, an agent is said to be randomly selected when the agent is chosen randomly without considering the specific sequences involved in the association of a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agents action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates. Dominant negative proteins, DNAs encoding these proteins, antibodies to these proteins, peptide fragments of these proteins or mimics of these proteins may be introduced into cells to affect function. “Mimic” used herein refers to the modification of a region or several regions of a peptide molecule to provide a structure chemically different from the parent peptide but topographically and functionally similar to the parent peptide (see Grant GA. in: Meyers (ed.) Molecular Biology and Biotechnology (New York, VCH Publishers, 1995), pp. 659-664). A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies as described above.

J. Uses for Agents that Modulate at Least One Activity of the Protein.

As provided in the Examples, the proteins and nucleic acids of the invention, such as the protein having the amino acid sequence of SEQ ID NO: 2, 4, 6, 8, 10, 12, 14, or 16, are differentially expressed during mast cell degranulation and/or in allergic hypersensitivity disease states. Agents that modulate or up-or-down-regulate the expression of the protein or agents, such as agonists or antagonists of at least one activity of the protein, may be used to modulate biological and pathologic processes associated with the protein's function and activity.

As used herein, a subject can be any mammal, so long as the mammal is in need of modulation of a pathological or biological process mediated by a protein of the invention. The term mammal is defined as an individual belonging to the class Mammalia. The invention is particularly useful in the treatment of human subjects.

Pathological processes refer to a category of biological processes which produce a deleterious effect. For example, expression of a protein of the invention may be associated with allergic hypersensitivity. As used herein, an agent is said to modulate a pathological process when the agent reduces the degree or severity of the process. For instance, allergic hypersensitivity may be prevented or disease progression modulated by the administration of agents which up- or down-regulate or modulate in some way the expression or at least one activity of a protein of the invention.

The agents of the present invention can be provided alone, or in combination with other agents that modulate a particular pathological process. For example, an agent of the present invention can be administered in combination with other known drugs. As used herein, two agents are said to be administered in combination when the two agents are administered simultaneously or are administered independently in a fashion such that the agents will act at the same time.

The agents of the present invention can be administered via parenteral, subcutaneous, intravenous, intramuscular, intraperitoneal, transdermal, or buccal routes. Alternatively, or concurrently, administration may be by the oral route. The dosage administered will be dependent upon the age, health, and weight of the recipient, kind of concurrent treatment, if any, frequency of treatment, and the nature of the effect desired.

The present invention further provides compositions containing one or more agents which modulate expression or at least one activity of a protein of the invention. While individual needs vary, determination of optimal ranges of effective amounts of each component is within the skill of the art. Typical dosages comprise 0.1 to 100 g/kg body wt. The preferred dosages comprise 0.1 to 10 g/kg body wt. The most preferred dosages comprise 0.1 to 1 g/kg body wt.

In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

In practicing the methods of this invention, the compounds of this invention may be used alone or in combination, or in combination with other therapeutic or diagnostic agents. In certain preferred embodiments, the compounds of this invention may be coadministered along with other compounds typically prescribed for these conditions according to generally accepted medical practice such as antihistamines. The compounds of this invention can be utilized in vivo, ordinarily in mammals, such as humans, sheep, horses, cattle, pigs, dogs, cats, rats and mice, or in vitro.

K. Transgenic Animals

Transgenic animals containing mutant, knock-out or modified genes corresponding to the cDNA sequence of SEQ ID NO: 1, 3, 5, 7, 9, 11, 13, or 15, or the open reading frame encoding the polypeptide sequence of SEQ ID NO: 2, 4, 6, 8, 10, 12, 14, or 16, or fragments thereof preferably having a consecutive sequence of at least about 25 or 30 amino acid residues, more preferably 35 or 40 amino acid residues, even more preferably 45 or 50 amino acid residues, yet more preferably 55 or 60, still more preferably 65 or 70 amino acid residues and most preferably at least 75 or more amino acid residues, are also included in the invention. Transgenic animals are genetically modified animals into which recombinant, exogenous or cloned genetic material has been experimentally transferred. Such genetic material is often referred to as a “transgene.” The nucleic acid sequence of the transgene, in this case a form of SEQ ID NO: 1, 3, 5, 7, 9, 11, 13, or 15 may be integrated either at a locus of a genome where that particular nucleic acid sequence is not otherwise normally found or at the normal locus for the transgene. The transgene may consist of nucleic acid sequences derived from the genome of the same species or of a different species than the species of the target animal.

The term “germ cell line transgenic animal” refers to a transgenic animal in which the genetic alteration or genetic information was introduced into a germ line cell, thereby conferring the ability of the transgenic animal to transfer the genetic information to offspring. If such offspring in fact possess some or all of that alteration or genetic information, then they too are transgenic animals.

The alteration or genetic information may be foreign to the species of animal to which the recipient belongs, foreign only to the particular individual recipient, or may be genetic information already possessed by the recipient. In the last case, the altered or introduced gene may be expressed differently than the native gene.

Transgenic animals can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al., (1993) Hypertension 22, 630-633; Brenin et al., (1997) Surg. Oncol. 6, 99-110; Tuan (1997) Recombinant Gene Expression Protocols, Methods in Molecular Biology, Humana Press).

A number of recombinant or transgenic mice have been produced, including those which express an activated oncogene sequence (U.S. Pat. No. 4,736,866); express simian SV40 T-antigen (U.S. Pat. No. 5,728,915); lack the expression of interferon regulatory factor 1 (IRF-1) (U.S. Pat. No. 5,731,490); exhibit dopaminergic dysfunction (U.S. Pat. No. 5,723,719); express at least one human gene which participates in blood pressure control (U.S. Pat. No. 5,731,489); display greater similarity to the conditions existing in naturally occurring Alzheimer's disease (U.S. Pat. No. 5,720,936); have a reduced capacity to mediate cellular adhesion (U.S. Pat. No. 5,602,307); possess a bovine growth hormone gene (Clutter et al., (1996) Genetics 143, 1753-1760); or, are capable of generating a fully human antibody response (McCarthy (1997) Lancet 349, 405).

While mice and rats remain the animals of choice for most transgenic experimentation, in some instances it is preferable or even necessary to use alternative animal species. Transgenic procedures have been successfully utilized in a variety of non-murine animals, including sheep, goats, pigs, dogs, cats, monkeys, chimpanzees, hamsters, rabbits, cows and guinea pigs (see, e.g., Kim et al., (1997) Mol. Reprod. Dev. 46, 515-526; Houdebine (1995) Reprod. Nutr. Dev. 35, 609-617; Petters (1994) Reprod. Fertil. Dev. 6, 643-645; Schnieke et al., (1997) Science 278, 2130-2133; and Amoah (1997) J. Animal Science 75, 578-585).

The method of introduction of nucleic acid fragments into recombination competent mammalian cells can be by any method which favors co-transformation of multiple nucleic acid molecules. Detailed procedures for producing transgenic animals are readily available to one skilled in the art, including the disclosures in U.S. Pat. No. 5,489,743 and U.S. Pat. No. 5,602,307.

L. Diagnostic Methods and Agents

The genes and proteins of the invention may be used to diagnose or monitor allergic hypersensitivity diseases, seasonal rhinitis, asthma, atopic dermatitis and amstocytosis or to track disease progression. One means of diagnosing allergic hypersensitivity using the nucleic acid molecules or proteins of the invention involves obtaining cells from skin, lung or respiratory tract tissue from living subjects. When possible, mucosal secretions, urine, blood or peripheral lymphocyte samples may be used as the tissue sample in the assay.

The use of molecular biological tools has become routine in forensic technology. For example, nucleic acid probes may be used to determine the expression of a nucleic acid molecule comprising all or at least part of the sequences of SEQ ID NO: 1, 3, 5, 7, 9, 11, 13, or 15 in forensic/pathology specimens. Further, nucleic acid assays may be carried out by any means of conducting a transcriptional profiling analysis. In addition to nucleic acid analysis, forensic methods of the invention may target the proteins of the invention, particularly a protein comprising SEQ ID NO: 2, 4, 6, 8, 10, 12, 14, or 16, to determine up or down regulation of the genes (Shiverick et al., (1975) Biochim Biophys Acta 393, 124-133).

Methods of the invention may involve treatment of tissues with collagenases or other proteases to make the tissue amenable to cell lysis (Semenov et al., (1987) Biull Eksp Biol Med 104, 113-116). Further, it is possible to obtain biopsy samples from different regions of the skin, respiratory tract or lungs for analysis.

Assays to detect nucleic acid or protein molecules of the invention may be in any available format. Typical assays for nucleic acid molecules include hybridization or PCR based formats. Typical assays for the detection of proteins, polypeptides or peptides of the invention include the use of antibody probes in any available format such as in situ binding assays, etc. See Harlow & Lane, (1988) Antibodies—A Laboratory Manual, Cold Spring Harbor Laboratory Press. In preferred embodiments, assays are carried-out with appropriate controls.

The above methods may also be used in other diagnostic protocols, including protocols and methods to detect disease states in other tissues or organs, for example the tissues in which gene expression is detected.

Without further description, it is believed that one of ordinary skill in the art can, using the preceding description and the following illustrative examples, make and utilize the compounds of the present invention and practice the claimed methods. The following working examples therefore, specifically point out preferred embodiments of the present invention, and are not to be construed as limiting in any way the remainder of the disclosure.

### EXAMPLES

### Example 1

Identification of the Differentially Expressed MC21 mRNA in Mast Cells

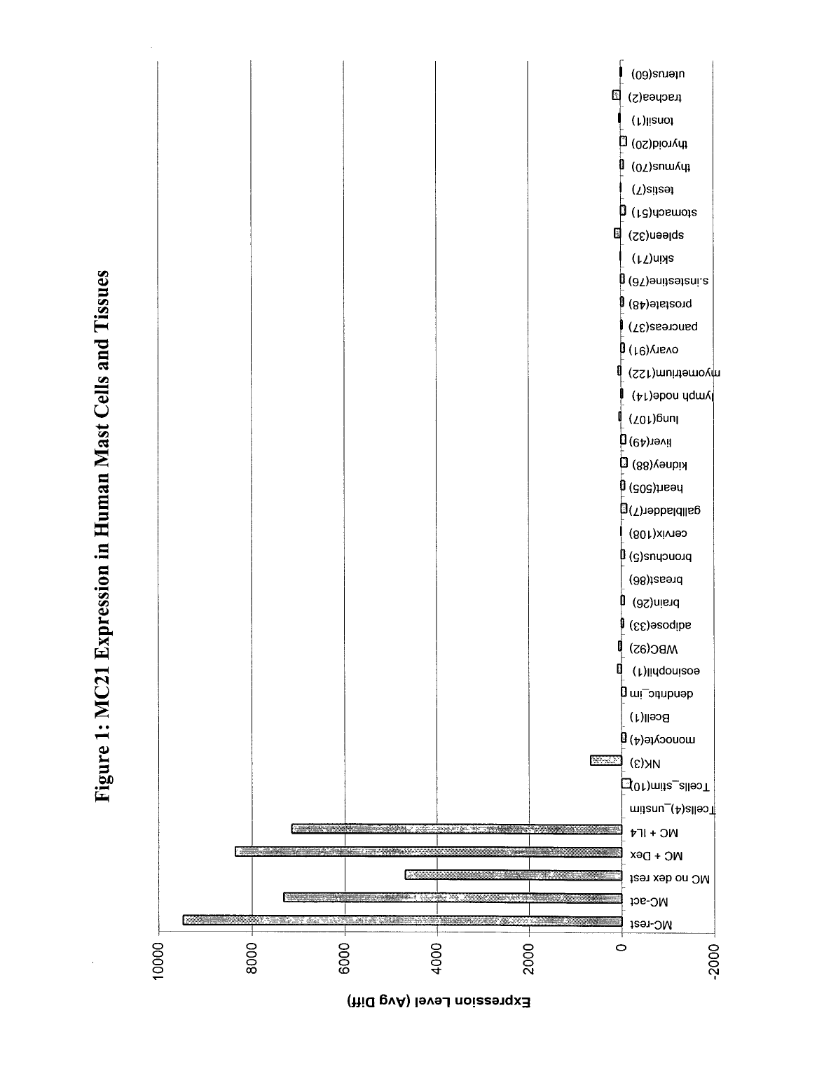

To identify genes which are differentially expressed or regulated in mast cells, mRNA was isolated from cultured human mast cells, isolated hematopoietic cells, and normal human tissues and used in an electronic Northern assay to probe a series of target sequences from the human genome. The intensity of expression of AF150143 (MC21; SEQ ID NO: 1) was measured across a panel of tissues and cell cultures using the Affymetrix human GeneChip 95K chip set. The mean intensity of expression is shown in FIG. 1. The number of data points included in each mean value is given in parentheses to the right of the tissue name. Mast cell samples were cultured for 6 weeks and then harvested or treated as specified. Rest=untreated. Act=3 hours after activation by IgE and antigen. Dex=24 hours after treatement by 10⁻⁷ M dexamethasone. Il-4=5 days after treatment with 10 ng/ml IL-4. T cells stim=stimulation by antibodies to CD3 and CD28.

### Example 2

Cloning of a Full Length Human cDNA Corresponding: to the Differentially Expressed mRNA species

The full-length cDNA having SEQ ID NO: 1 was obtained by the solution hybridization method. Briefly, a gene-specific oligo was designed based on the sequence of the EST fragment identified in Example 1. The oligo was labeled with biotin and used to hybridize with 5 μg of single strand plasmid DNA (cDNA recombinants) from a human resting mast cell library following the procedures from the GeneTrapper® kit obtained from Invitrogen. The hybridized cDNAs were separated by streptavidin-conjugated beads and eluted by TE buffer. The eluted cDNA was converted to double strand plasmid DNA and used to transform E. Coli cells (DH 5).

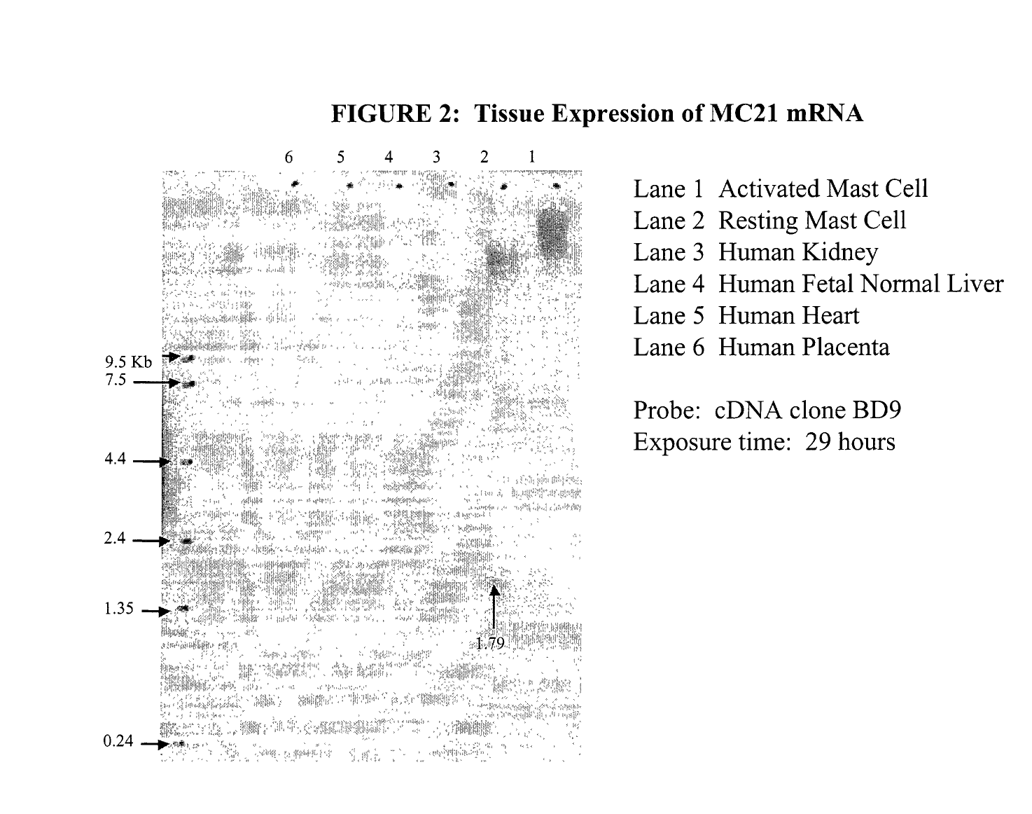

The nucleotide sequences of the full-length human cDNA corresponding to the differentially regulated mRNAs detected above is set forth in SEQ ID NO: 1. The cDNA comprises 1596 base pairs, with a open reading frame at nucleotides 70-1038 (nucleotides 70-1041 with the TAA stop codon) encoding a protein of 323 amino acids. The amino acid sequence corresponding to this encoded protein is set forth in SEQ ID NO: 2. FIG. 2 shows a Northern blot using MC21 cDNA clone BD9 as probe, in which the expression level of a 1.79 kb mRNA species corresponding to SEQ ID NO: 1 was measured in human cultured mast cells or tissues. Lane 1) activated mast cells, Lane 2) resting mast cells, Lane 3) kidney, Lane 4) fetal liver, Lane 5) heart, Lane 6) placenta. A probe based on SEQ ID NO: 1 (randomly labeled, using the Stratagene PRIME-IT II kit) was exposed to human mRNA blots (ClonTech mRNA blot-H4) using sensitive hybridization conditions (Church-Gilbert buffer, 29 hours at room temperature). Significant expression of a 1.79 Kb species was seen in the resting mast cell sample and lower levels were seen in the activated mast cells.

### Example 3

Detection of Allergic Hypersensitivity in a Patient

The expression level of a nucleic acid or protein of the invention is determined in a sample from a patient suspected of allergic hypersensitivity before and/or after exposure to a potential antigen. The sample may be from an epithelial tissue such as a skin, respiratory tract or lung cell samples, or in urine or in blood samples. The sample may also be bronchoalveloar wash. Tissue or isolated mast cell samples from a patient known to have allergic hypersensitivity and from normal subjects may be used as positive and negative controls, respectively. A change in the level of expression of the nucleic acid or protein of the invention compared to the expression level in control or normal tissue may be indicative of allergic hypersensitivity.

### Example 4

Hydrophobicity and Antigenicity of MC21

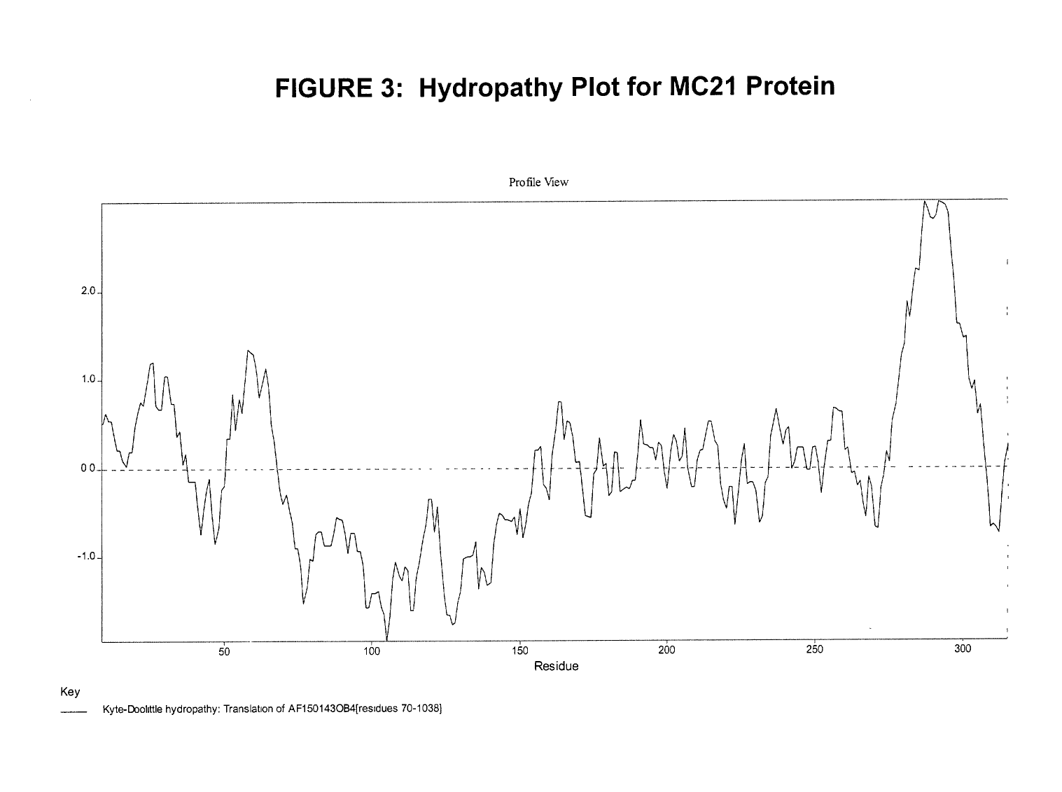

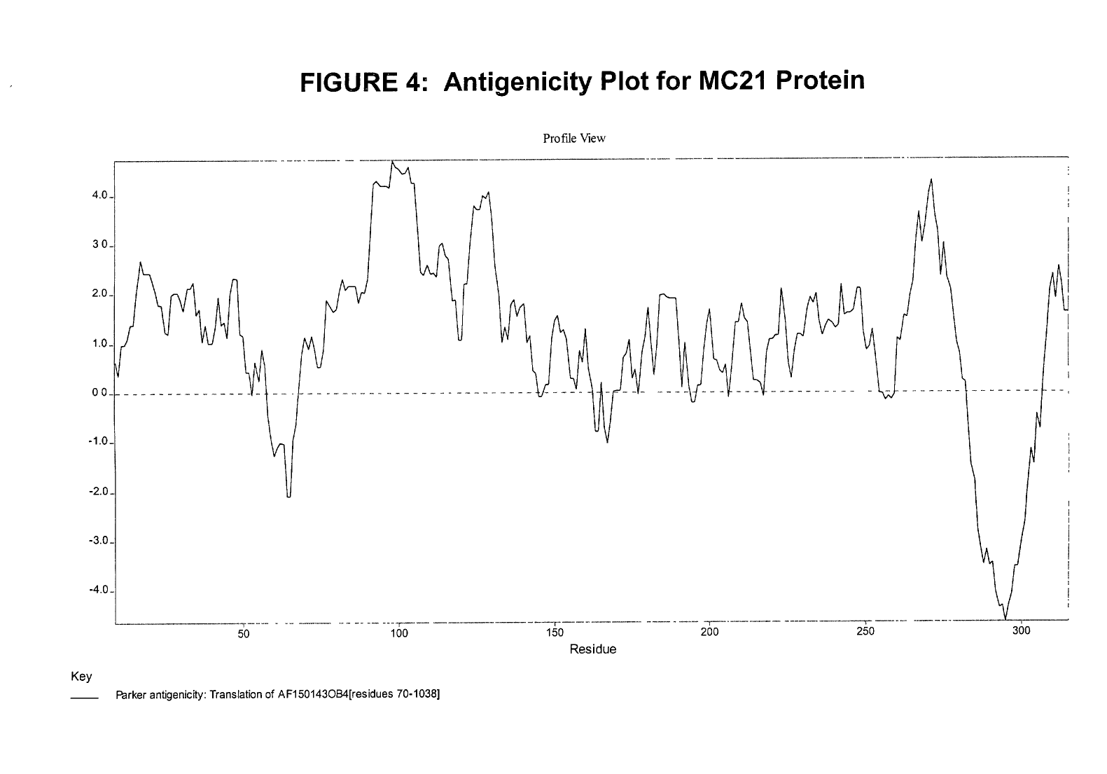

The translation of the open reading frame of cDNA clone OB4, residues 70-1038 of SEQ ID NO: 2, which corresponds to the predicted full-length protein sequence of MC21 (SEQ ID NO: 2) was analyzed by the Kile-Doolittle method for hydrophobic regions within the protein (FIG. 3). The sequence was further analyzed using the Parker method for the presence of particular antigenic regions or epitopes (FIG. 4).

### Example 5

Identification of Additional Differentially Expressed mRNAs in Mast Cells

Several additional genes were identified which were differentially expressed in mast cells versus other human cell types and tissues. In an electronic Northern assay, the Affymetrix human GeneChip 95K chip set was probed using mRNA samples from cultured human mast cells, isolated hematopoietic cells, and normal human tissues. cDNAs were obtained by multiple methods. CDNAs for MC25 and MC39 were obtained by solution hybridization as in Example 2. cDNAs for MC22, MC33, and MC36 were obtained by 5′ RACE from the est sequence to determine the 5′ end. This sequence as well as human genomic sequence information was used to design primers to PCR the cDNA from mast cell RNA.

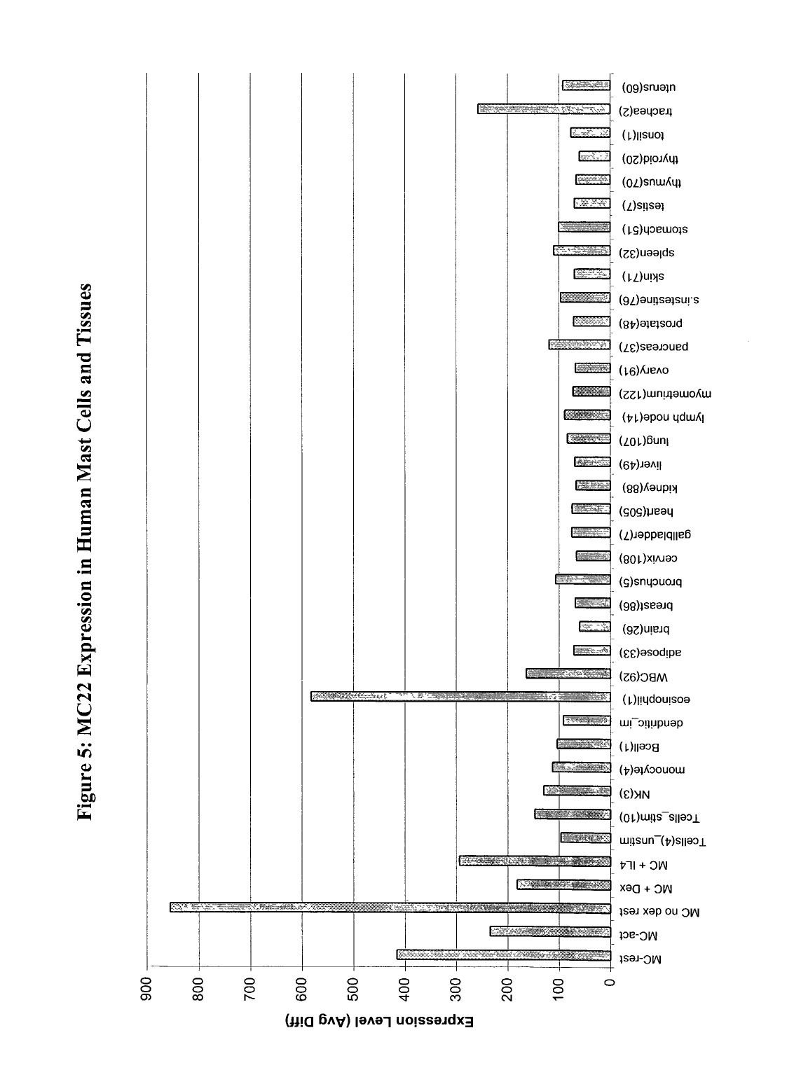

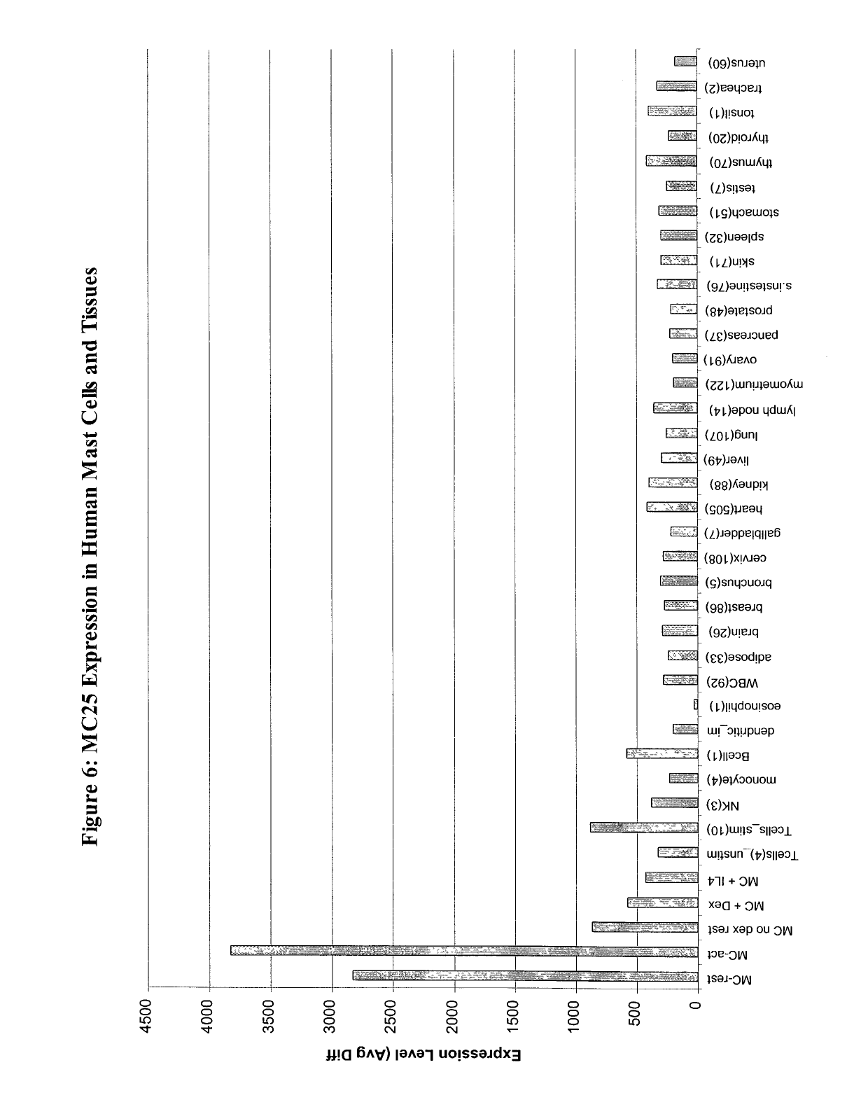

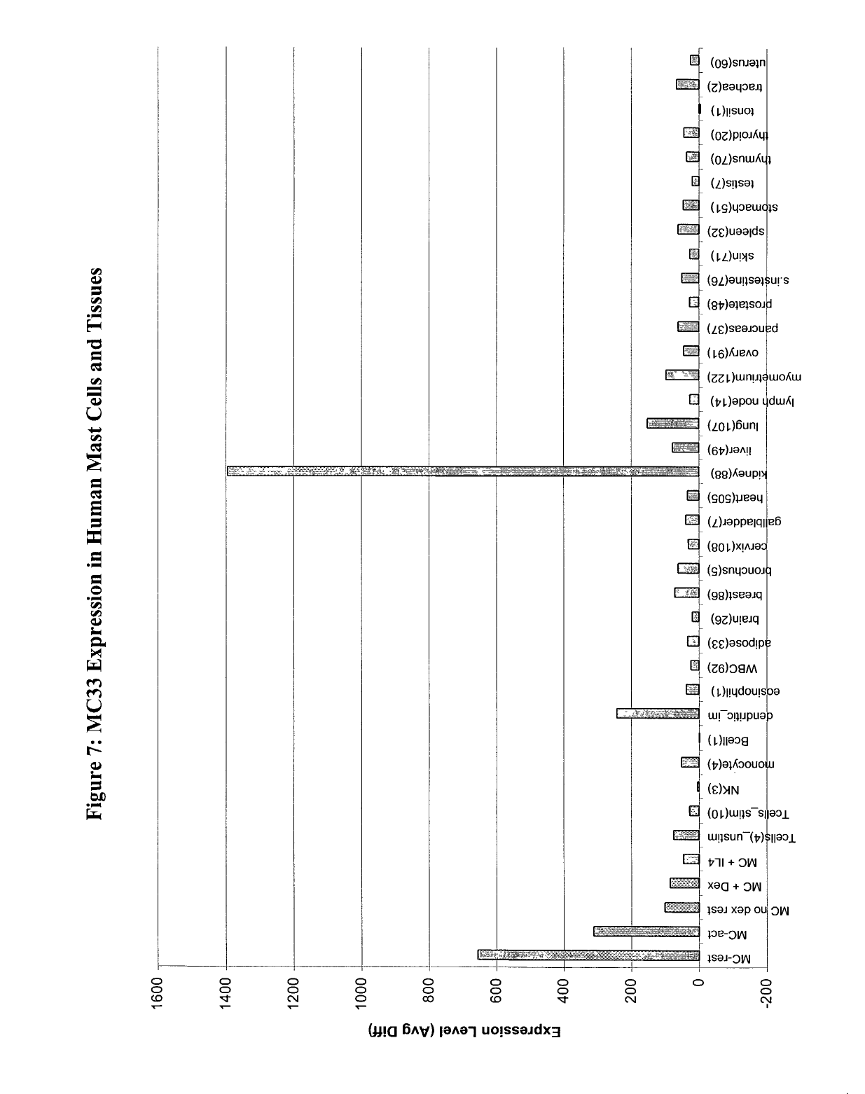

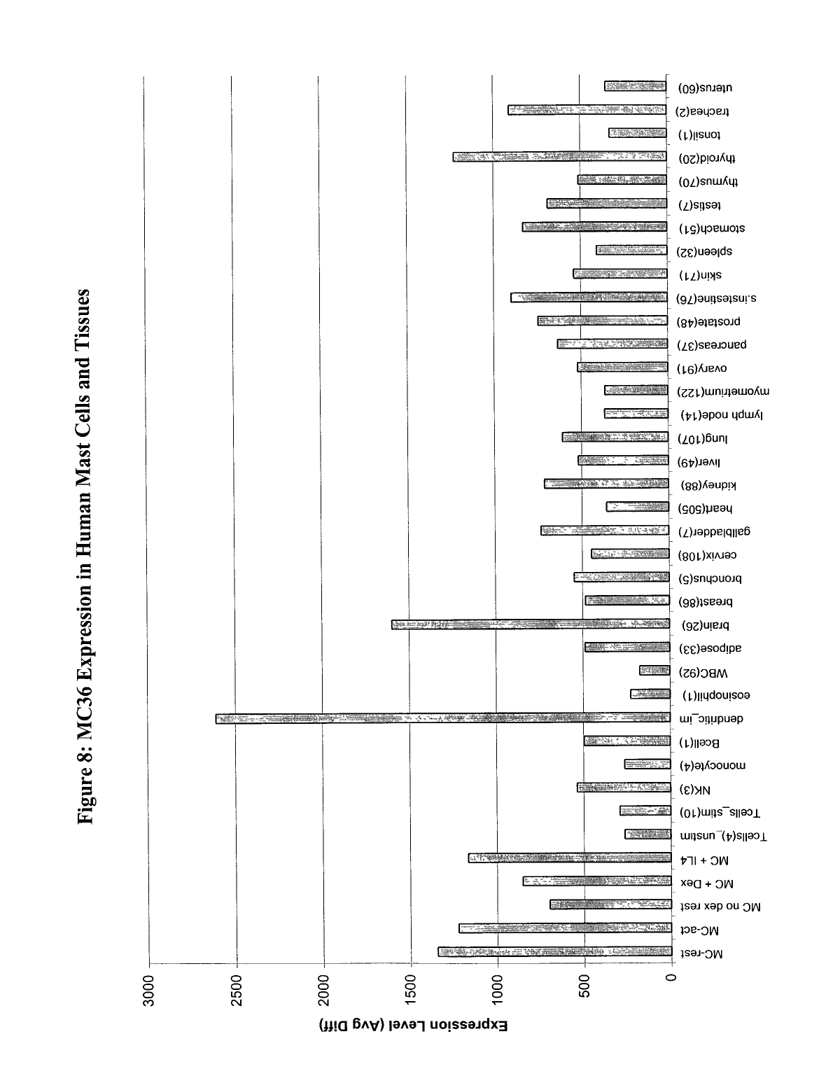

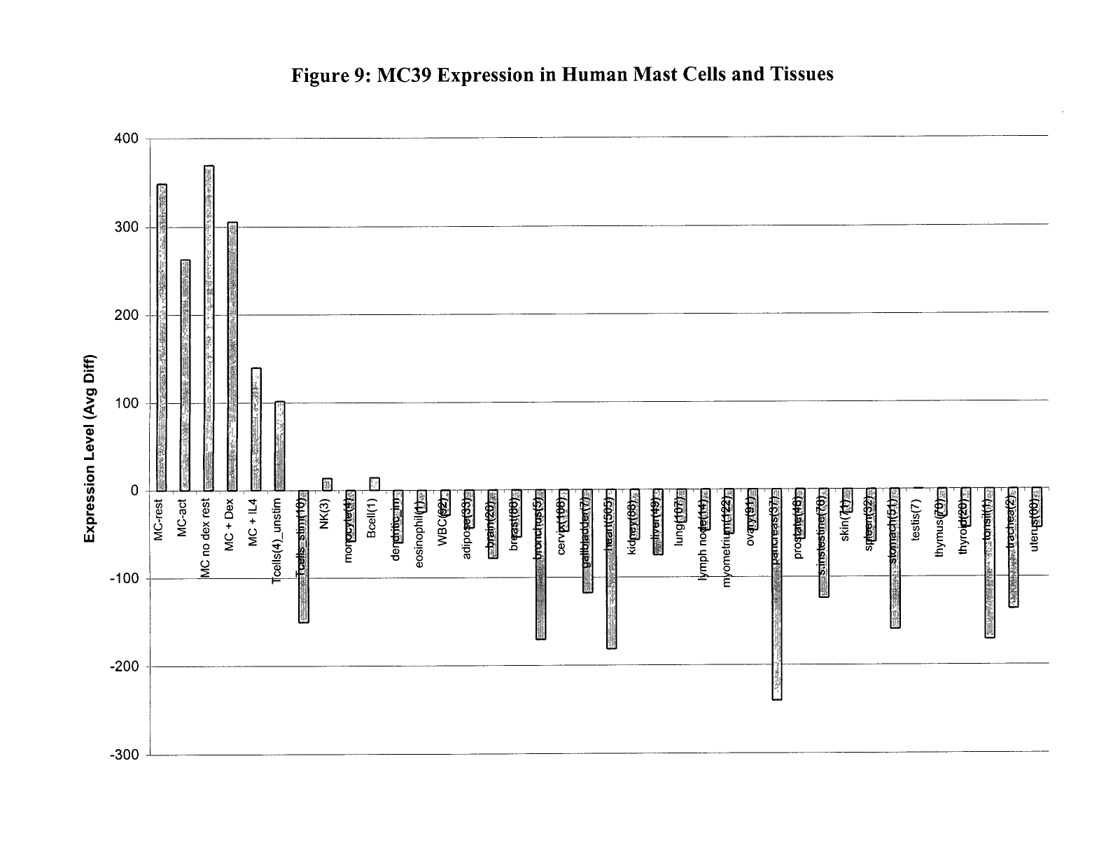

FIG. 5 shows the intensity of expression of MC22. SEQ ID NOs: 3 and 5 represent splice-variants of the full-length human cDNA corresponding to the differentially expressed mRNAs detected, differing from one another by the insert at nucleotides 2147-2254 of SEQ ID NO: 3. FIG. 6 shows the intensity of expression of MC25 (SEQ ID NO: 7), FIG. 7 shows the intensity of expression of MC33 (SEQ ID NO: 9), and FIG. 8 shows the intensity of expression of MC36 (SEQ ID NO: 11). FIG. 9 shows the intensity of expression of MC39, which is represented by the variants of SEQ ID NOs: 13 and 15).

For each figure, the mean intensity of expression, measured using the Affymetrix human GeneChip 95K chip set, is shown. The number of data points included in each mean value is given in parentheses to the right of the tissue name. Mast cell samples were cultured for 6 weeks and then harvested or treated as specified. Rest=untreated. Act=3 hours after activation by IgE and antigen. Dex=24 hours after treatment by 10-7 M dexamethasone. Il-4=5 days after treatment with 10 ng/ml IL-4. T cells stim=stimulation by antibodies to CD3 and CD28.

A summary of the genes identified here as being differentially expressed and/or regulated in human mast cells is presented in the Table.

| Mast Cell | DNA | Protein | | | |
|---|---|---|---|---|---|
| Gene | Seq | Seq | Affymetrix | Affymetrix | |
| Name | ID NO: | ID NO: | Identifier | CHIP/ID | Description |
| MC21 | 1 | 2 | AF150143 | U95/78123 | siglec-like |
| | | | | | receptor |
| MC22 | 3, 5 | 4, 6 | AI911742 | U95/71487 | homologous |
| | | | | | to rat |
| | | | | | Tomosyn |
| MC25 | 7 | 8 | AI631846 | U95/75150 | kelch |
| | | | | | domain |
| MC33 | 9 | 10 | AI733048 | U95/90676 | homologous |
| | | | | | to V ATPase |
| | | | | | D subunit |
| MC36 | 11 | 12 | AL117442 | U95/49047 | acyl |
| | | | | | transferase, |
| | | | | | Ca binding |
| MC39 | 13, 15 | 14, 16 | AA932726 | U95/83815 | poly proline |

Although the present invention has been described in detail with reference to examples above, it is understood that various modifications can be made without departing from the spirit of the invention. Accordingly, the invention is limited only by the following claims. All cited patents, patent applications and publications referred to in this application are herein incorporated by reference in their entirety.

What is claimed:

1. An isolated nucleic acid molecule selected from the group consisting of: (a) an isolated nucleic acid molecule that encodes the amino acid sequence of SEQ ID NO: 2, 4, 6, 8, 10, 12, 14, or 16; (b) an isolated nucleic acid molecule which hybridizes under stringent conditions to the complement of SEQ ID NO: 1, 3, 5, 7, 9, 11, 13, or 15; (c) an isolated nucleic acid molecule which hybridizes under stringent conditions to the complement of a nucleic acid molecule that encodes the amino acid sequence of SEQ ID NO: 2, 4, 6, 8, 10, 12, 14, or 16; (d) an isolated nucleic acid molecule that encodes a protein that exhibits at least about 90% amino acid sequence identity to SEQ ID NO: 2, 4, 6, 8, 10, 12, 14, or 16; and (e) an isolated nucleic acid molecule comprising the complement of SEQ ID NO: 1, 3, 5, 7, 9, 11, 13, or 15.

2. The isolated nucleic acid molecule of claim 1, wherein the nucleic acid molecule comprises nucleotides 70-1041 of SEQ ID NO: 1.

3. The isolated nucleic acid molecule of claim 1, wherein the nucleic acid molecule consists of nucleotides 70-1038 of SEQ ID NO: 1.

4. The isolated nucleic acid molecule of claim 1, wherein the nucleic acid molecule encodes the amino acid sequence of SEQ ID NO: 2.

5. The isolated nucleic acid molecule of claim 1, wherein the nucleic acid molecule encodes the mature protein of MC21 or amino acid residues 39-323 of SEQ ID NO: 2.

6. The isolated nucleic acid molecule of claim 1, wherein the nucleic acid molecule encodes the extracellular domain of MC21 or amino acid residues 39-278 of SEQ ID NO: 2.

7. The isolated nucleic acid molecule of claim 1, wherein the nucleic acid molecule encodes the extracellular domain of MC21 contiguous with the transmembrane domain of MC21 or amino acid residues 39-278 of SEQ ID NO: 2.

8. The isolated nucleic acid molecule of claim 1, wherein said nucleic acid molecule is operably linked to one or more expression control elements.

9. A vector comprising an isolated nucleic acid molecule of claim 1.

10. A host cell transformed to contain the nucleic acid molecule of claim 1.

11. A host cell comprising the vector of claim 9.

12. The host cell of claim 11, wherein said host is selected from the group consisting of prokaryotic host cells and eukaryotic host cells.

13. A method for producing a polypeptide comprising culturing a host cell transformed with the nucleic acid molecule of claim 1 under conditions in which the protein encoded by said nucleic acid molecule is expressed.

14. The method of claim 13, wherein said host cell is selected from the group consisting of prokaryotic host cells and eukaryotic host cells.

15. An isolated polypeptide produced by the method of claim 13.

16. An isolated antibody that binds to a polypeptide of claim 15.

17. An isolated polypeptide or protein selected from the group consisting of an isolated polypeptide comprising the amino acid sequence of SEQ ID NO: 2, 4, 6, 8, 10, 12, 14, or 16, an isolated polypeptide comprising a fragment of at least 6 amino acids of SEQ ID NO: 2, 4, 6, 8, 10, 12, 14, or 16, an isolated polypeptide comprising conservative amino acid substitutions of SEQ ID NO: 2, 4, 6, 8, 10, 12, 14, or 16 or naturally occurring amino acid sequence variants of SEQ ID NO: 2, 4, 6, 8, 10, 12, 14, or 16, and an isolated polypeptide exhibiting at least about 90% amino acid sequence identity with SEQ ID NO: 2, 4, 6, 8, 10, 12, 14, or 16.

18. An isolated polypeptide or protein of claim 16, selected from the group consisting of an isolated polypeptide comprising the amino acid sequence of SEQ ID NO: 2, an isolated polypeptide comprising mature MC21 protein or amino acid residues 39-323 of SEQ ID NO: 2, an isolated polypeptide comprising MC21 extracellular domain amino acid residues 39-278 of SEQ ID NO: 2, an isolated polypeptide comprising the extracellular domain of MC21 contiguous to the transmembrane domain of MC21 protein, an isolated polypeptide comprising conservative amino acid substitutions of SEQ ID NO: 2 or naturally occurring amino acid sequence variants of SEQ ID NO: 2, and an isolated polypeptide exhibiting at least about 90% amino acid sequence identity with SEQ ID NO: 2.

19. An isolated antibody that binds to a polypeptide of claim 17.

20. An antibody of claim 19 wherein said antibody is a monoclonal or a polyclonal antibody.

21. A method of identifying an agent which modulates the expression of a nucleic acid encoding a protein of claim 17, comprising:
exposing cells which express the nucleic acid to the agent; and determining whether the agent modulates expression of said nucleic acid, thereby identifying an agent which modulates the expression of a nucleic acid encoding the protein.

22. A method of identifying an agent which modulates at least one activity of a protein of claim 17, comprising:
exposing cells which express the protein to the agent;
determining whether the agent modulates at least one activity of said protein, thereby identifying an agent which modulates at least one activity of the protein.

23. The method of claim 21, wherein the agent modulates one activity of the protein.

24. A method of identifying binding partners for a protein of claim 17, comprising:
exposing said protein to a potential binding partner; and
determining if the potential binding partner binds to said protein, thereby identifying binding partners for the protein.

25. A method of modulating the expression of a nucleic acid encoding a protein of claim 17, comprising:
administering an effective amount of an agent which modulates the expression of a nucleic acid encoding the protein.

26. A method of modulating at least one activity of a protein of claim 17, comprising:
administering an effective amount of an agent which modulates at least one activity of the protein.

27. A non-human transgenic animal modified to contain the nucleic acid molecule of claim 1.

28. The transgenic animal of claim 27, wherein the nucleic acid molecule contains a mutation that prevents expression of the encoded protein.

29. A method of diagnosing a disease state in a subject, comprising determining the level of expression of a nucleic acid molecule of claim 1.

30. The method of claim 29, wherein the disease state is allergic hypersensitivity.

31. The method of claim 29, wherein the disease state is seasonal rhinitis.

32. The method of claim 29, wherein the disease state is asthma.

33. The method of claim 29, wherein the disease state is urticaria or atopic dermatitis.

34. The method of claim 29, wherein the disease state is mastocytosis.

35. A composition comprising an isolated nucleic acid molecule of claim 1 and an aqueous carrier.

36. A method of diagnosing a disease state in a subject, comprising determining the level of expression of a protein of claim 17.

37. The method of claim 36, wherein the disease state is allergic hypersensitivity.

38. The method of claim 36, wherein the disease state is seasonal rhinitis.

39. The method of claim 36, wherein the disease state is asthma.

40. The method of claim 36, wherein the disease state is urticaria or atopic dermatitis.

41. The method of claim 37, wherein the disease state is mastocytosis.


SEQUENCE LISTING (16 sequences)

SEQ ID NO: 1 - DNA, 1596 bp, Homo sapiens
  CDS (70)..(1041)
gtgcagaggcggatgacctctgaccacgtgtgggtttgaggttcctcttctgtgaactgccaagatctcatgctcctccccacagccctcttctctgctcacacaggaagcccaggaagcctctgcctcagagatgctgccgctgctgctgccgctgcccctgctgtgggcagggccctcgctcaggatgcaagattccggctggagatgccagagtccgtgacggtgcaggagggtctgtgcatctttgtgcactgttcggtcttctacctcgagtatggctggaaagattctacccctgcttatggccactggttccgggaaggggtcagtgtagaccaggagactccagtggccacaaacaactcaactcaaaaagtgcagaaggagacccagggccgattccacctcctcggtgatccctcaaggaacaactgctccctgagcatcagagacgccaggaggagggacaacggttcatacttcttttgggtggcgagaggaagaacaaaatttagttacaaatattccccgctctctgtgtatgtgacagccctgacccacaggcccgacatcctcatcccggagttcctaaagtctggccatcccagcaacctgacctgctctgtgccctgggtctgtgagcagggaacaccccccatcttctcctggatgtcagctgcccccacctccctgggccccaggaccctccactcctcagtgctcacgatcatcccacggcctcaggaccacggcaccaacctcatctgtcaggtgacgttccccggagctggtgtgaccacggagagaaccatccagctcagtgtctcctatcctctaatgaacccagtgattgaagcctccctagaagacggcacagggaaatcaggaaccgtggaagaggtggttgttttggccgtgggggtagtggctgtgaagatcctgcttctctgcctttgcctcatcatcctcagtttccacaagaagaaggcggtgagggcagtggaggttgaggagaatgtatatgctgtcatgggttaatctctcaggcctccagactgtacttccagatgtctcctcatccagttcctccacagtctgaatggccatgtttcttcttcattgctggagaatgaagtgcaaatgccactgcctggactgaaggcctttcacgatctgtcttctgctggactctgctcctgatcccccttctccttgcatcacccgaagtctccctacacccaccaggccaagccctctgtgattctgagactttgcatgtgtagttacttctcctgaaatggccttcctccccattcctgccaatccaggtccttatcatccttcaggttgtcttaaatgtcatccaggtgtgtgtatttttatgtaatccttgtatgatattaagcggagatgtggcatttgttcattaatttgtagacatattcagtaaccatactgaatacatataatgactatgtgccagcatttccgtatgtgcaagaagttcatcaatagatatagactcaaagagctctgtcatcaagctgttgttctgaagagcagaaggatacaaataaaaagaaataagtaaaa

SEQ ID NO: 2 - protein, 323 aa, Homo sapiens
MLLPTALFSAHTGSPGSLCLRDAAAAAAAAPAVGRALAQDARFRLEMPESVTVQEGLCIFVHCSVFYLEYGWKDSTPAYGHWFREGVSVDQETPVATNNSTQKVQKETQGRFHLLGDPSRNNCSLSIRDARRRDNGSYFFWVARGRTKFSYKYSPLSVYVTALTHRPDILIPEFLKSGHPSNLTCSVPWVCEQGTPPIFSWMSAAPTSLGPRTLHSSVLTIIPRPQDHGTNLICQVTFPGAGVTTERTIQLSVSYPLMNPVIEASLEDGTGKSGTVEEVVVLAVGVVAVKILLLCLCLIILSFHKKKAVRAVEVEENVYAVMG

SEQ ID NO: 3 - DNA, 6912 bp, Homo sapiens
  CDS (1)..(3456)
atgaggaaattcaacatcaggaaggtgctggacggcctgaccgccggctcgtcctcggcgtcgcagcagcaacagcagcagcatccgcctgggaaccgggagccggagatccaggaaacgctccagtccgagcactttcagctctgcaagactgttcgccatggatttccctatcaaccctcagccctggcctttgatcctgtacagaagatcctggcagtgggaactcagactggtgctttaaggctctttggtcgtccaggagtagaatgttattgccagcatgacagtggagctgcagtaatccagctccagttcctgattaatgagggagcgcttgtgagtgccttggctgatgacaccttacacttatggaatttacgtcagaagaggcctgccatactacattcgcttaaattttgcagagaaagggttacattttgccatctgcctttccagagtaagtggctctatgtgggcactgaacgaggtaatatacatattgtcaatgtggagtccttcacactctcaggctacgtcattatgtggaataaagccattgaactgtcatctaaatctcacccaggacctgtggtccatataagtgataatccaatggacgagggaaagcttttgattggctttgaatctggaacagtagttttatgggacctcaaatcaaagaaagccgactacagatacacatatgatgaggctatccactctgttgcttggcatcatgaaggaaaacaatttatttgcagtcattcagatggcaccttgactatatggaatgtaaggtcccctgctaaaccagtacagacaatcactccacatggaaaacagttaaaggatgggaagaagccagaaccatgcaaacctatcctcaaggtggaattcaaaacgactagatctggggagccttttattattttatcaggaggtttgtcatatgatactgtaggaagaagaccttgcttaacagtgatgcatgggaaaagcactgctgtgctagaaatggactattcaattgttgattttctaacgctgtgtgaaacaccatacccaaatgattttcaagaaccatatgctgtggttgttcttctagaaaaggatttagtacttatagaccttgcacaaaatggatatcctatatttgaaaatccctaccctttgagtatacatgagtcccctgttacatgttgcgaatattttgcggattgtcctgtggaccttattcctgcactttattctgttggagctagacagaaacgtcaaggttacagcaaaaaggaatggcccatcaacggaggtaattggggcttgggtgctcaaagttacccagaaataattattacagggcatgctgatgggtcagttaagttctgggatgcttctgcaataactctacaagtattatataagctaaagacatctaaagtatttgaaaagtcaagaaataaagatgacaggccaaacacagacattgtagatgaagatccatatgccattcagatcatctcctggtgtccagaaagtagaatgctgtgcatcgctggagtttcagctcatgtcattatttatagattcagcaagcaggaagtaatcacagaagtcattccgatgcttgaagttcgattattatatgagataaatgatgtggaaactccggagggtgagcagccaccacctttgccaacacccgtgggagggtccaaccctcagcccatccctcctcagtctcatccatctaccagtagcagttcatctgatgggcttcgtgataatgtaccttgtttaaaagttaaaaactcaccacttaaacagtctccaggttatcaaacagaactagttattcagttggtttgggtgggtggagaaccaccacaacaaataaccagcctggcagtcaattcttcctatggactggtggtttttggcaattgcaatggcattgctatggttgactacctccagaaagcagtgctgctcaacctgggcactattgaattatatggctctaatgatccttatcggagagaaccccgatctcctcgtaaatctcgacagccttcaggagccggtctgtgtgatattagtgaagggactgttgttccagaggatcgctgcaaatctccaacctctggttcttcatcaccacacaattcagatgatgaacaaaaaatgaataattttatagaaaaggtgaagaccaaaagcagaaagttttccaagatggtagccaatgatatagcaaagatgtcaaggaagttaagcttacctactgacctaaagcctgatttagatgtaaaggataactcctttagccgatcacggagttcaagtgtaacaagcattgacaaagaatcccgagaagcgatctccgctcttcatttctgtgaaacgtttactcgaaagacggactcgtccccttccccttgtctgtgggttggaacaacgctaggaacagtgcttgtcattgcactgaaccttcccccagggggagagcaaagacttcttcagccagtaattgtgtctccaagtggtactatattgaggttaaaaggtgcaatcttgagaatggcatttctggataccacaggctgcttaataccacctgcgtatgaaccctggagagagcacaatgttcctgaagaaaaagacgaaaaggagaaattgaaaaaacggcggcctgtctcagtatccccctcctcttctcaggaaattagtgaaaaccagtatgcagtgatatgttctgaaaagcaagcaaaagtaatctcactgccaacccagaactgtgcttataagcaaaatattacagagacctcgtttgtgcttcgtggagatattgtagcattgagtaacagtatctgccttgcctgtttctgtgccaatggacatataatgacttttagtttgccaagtttaagacctctgttggatgtgtattacttgccccttaccaatatgcggatagccagaacgttctgctttaccaacaatggacaagcattataccttgtttcacctacagaaatccagagacttacttatagtcaagagacctgtgaaaatcttcaggaaatgttgggtgaactcttcactcctgtagaaacacctgaagcaccaaacaggggattctttaaaggcttatttggaggtggtgcacaatctcttgacagagaagaactatttggagaatcgtcctcaggaaaggcttcaaggagccttgcacagcatattcctggccctggtggcattgaaggcgtaaaaggggcagcatctggagttgttggtgaattagcacgagccaggctggcactagatgaaagagggcagaaacttggcgatctggaagaaagaactgcggccatgttatcaagtgcagagtcattttctaaacatgctcatgagattatgttgaaatacaaagataagaagtggtaccagttctgaatgaggaaattcaacatcaggaaggtgctggacggcctgaccgccggctcgtcctcggcgtcgcagcagcaacagcagcagcatccgcctgggaaccgggagccggagatccaggaaacgctccagtccgagcactttcagctctgcaagactgttcgccatggatttccctatcaaccctcagccctggcctttgatcctgtacagaagatcctggcagtgggaactcagactggtgctttaaggctctttggtcgtccaggagtagaatgttattgccagcatgacagtggagctgcagtaatccagctccagttcctgattaatgagggagcgcttgtgagtgccttggctgatgacaccttacacttatggaatttacgtcagaagaggcctgccatactacattcgcttaaattttgcagagaaagggttacattttgccatctgcctttccagagtaagtggctctatgtgggcactgaacgaggtaatatacatattgtcaatgtggagtccttcacactctcaggctacgtcattatgtggaataaagccattgaactgtcatctaaatctcacccaggacctgtggtccatataagtgataatccaatggacgagggaaagcttttgattggctttgaatctggaacagtagttttatgggacctcaaatcaaagaaagccgactacagatacacatatgatgaggctatccactctgttgcttggcatcatgaaggaaaacaatttatttgcagtcattcagatggcaccttgactatatggaatgtaaggtcccctgctaaaccagtacagacaatcactccacatggaaaacagttaaaggatgggaagaagccagaaccatgcaaacctatcctcaaggtggaattcaaaacgactagatctggggagccttttattattttatcaggaggtttgtcatatgatactgtaggaagaagaccttgcttaacagtgatgcatgggaaaagcactgctgtgctagaaatggactattcaattgttgattttctaacgctgtgtgaaacaccatacccaaatgattttcaagaaccatatgctgtggttgttcttctagaaaaggatttagtacttatagaccttgcacaaaatggatatcctatatttgaaaatccctaccctttgagtatacatgagtcccctgttacatgttgcgaatattttgcggattgtcctgtggaccttattcctgcactttattctgttggagctagacagaaacgtcaaggttacagcaaaaaggaatggcccatcaacggaggtaattggggcttgggtgctcaaagttacccagaaataattattacagggcatgctgatgggtcagttaagttctgggatgcttctgcaataactctacaagtattatataagctaaagacatctaaagtatttgaaaagtcaagaaataaagatgacaggccaaacacagacattgtagatgaagatccatatgccattcagatcatctcctggtgtccagaaagtagaatgctgtgcatcgctggagtttcagctcatgtcattatttatagattcagcaagcaggaagtaatcacagaagtcattccgatgcttgaagttcgattattatatgagataaatgatgtggaaactccggagggtgagcagccaccacctttgccaacacccgtgggagggtccaaccctcagcccatccctcctcagtctcatccatctaccagtagcagttcatctgatgggcttcgtgataatgtaccttgtttaaaagttaaaaactcaccacttaaacagtctccaggttatcaaacagaactagttattcagttggtttgggtgggtggagaaccaccacaacaaataaccagcctggcagtcaattcttcctatggactggtggtttttggcaattgcaatggcattgctatggttgactacctccagaaagcagtgctgctcaacctgggcactattgaattatatggctctaatgatccttatcggagagaaccccgatctcctcgtaaatctcgacagccttcaggagccggtctgtgtgatattagtgaagggactgttgttccagaggatcgctgcaaatctccaacctctggttcttcatcaccacacaattcagatgatgaacaaaaaatgaataattttatagaaaaggtgaagaccaaaagcagaaagttttccaagatggtagccaatgatatagcaaagatgtcaaggaagttaagcttacctactgacctaaagcctgatttagatgtaaaggataactcctttagccgatcacggagttcaagtgtaacaagcattgacaaagaatcccgagaagcgatctccgctcttcatttctgtgaaacgtttactcgaaagacggactcgtccccttccccttgtctgtgggttggaacaacgctaggaacagtgcttgtcattgcactgaaccttcccccagggggagagcaaagacttcttcagccagtaattgtgtctccaagtggtactatattgaggttaaaaggtgcaatcttgagaatggcatttctggataccacaggctgcttaataccacctgcgtatgaaccctggagagagcacaatgttcctgaagaaaaagacgaaaaggagaaattgaaaaaacggcggcctgtctcagtatccccctcctcttctcaggaaattagtgaaaaccagtatgcagtgatatgttctgaaaagcaagcaaaagtaatctcactgccaacccagaactgtgcttataagcaaaatattacagagacctcgtttgtgcttcgtggagatattgtagcattgagtaacagtatctgccttgcctgtttctgtgccaatggacatataatgacttttagtttgccaagtttaagacctctgttggatgtgtattacttgccccttaccaatatgcggatagccagaacgttctgctttaccaacaatggacaagcattataccttgtttcacctacagaaatccagagacttacttatagtcaagagacctgtgaaaatcttcaggaaatgttgggtgaactcttcactcctgtagaaacacctgaagcaccaaacaggggattctttaaaggcttatttggaggtggtgcacaatctcttgacagagaagaactatttggagaatcgtcctcaggaaaggcttcaaggagccttgcacagcatattcctggccctggtggcattgaaggcgtaaaaggggcagcatctggagttgttggtgaattagcacgagccaggctggcactagatgaaagagggcagaaacttggcgatctggaagaaagaactgcggccatgttatcaagtgcagagtcattttctaaacatgctcatgagattatgttgaaatacaaagataagaagtggtaccagttctga

SEQ ID NO: 4 - protein, 1151 aa, Homo sapiens
MRKFNIRKVLDGLTAGSSSASQQQQQQHPPGNREPEIQETLQSEHFQLCKTVRHGFPYQPSALAFDPVQKILAVGTQTGALRLFGRPGVECYCQHDSGAAVIQLQFLINEGALVSALADDTLHLWNLRQKRPAILHSLKFCRERVTFCHLPFQSKWLYVGTERGNIHIVNVESFTLSGYVIMWNKAIELSSKSHPGPVVHISDNPMDEGKLLIGFESGTVVLWDLKSKKADYRYTYDEAIHSVAWHHEGKQFICSHSDGTLTIWNVRSPAKPVQTITPHGKQLKDGKKPEPCKPILKVEFKTTRSGEPFIILSGGLSYDTVGRRPCLTVMHGKSTAVLEMDYSIVDFLTLCETPYPNDFQEPYAVVVLLEKDLVLIDLAQNGYPIFENPYPLSIHESPVTCCEYFADCPVDLIPALYSVGARQKRQGYSKKEWPINGGNWGLGAQSYPEIIITGHADGSVKFWDASAITLQVLYKLKTSKVFEKSRNKDDRPNTDIVDEDPYAIQIISWCPESRMLCIAGVSAHVIIYRFSKQEVITEVIPMLEVRLLYEINDVETPEGEQPPPLPTPVGGSNPQPIPPQSHPSTSSSSSDGLRDNVPCLKVKNSPLKQSPGYQTELVIQLVWVGGEPPQQITSLAVNSSYGLVVFGNCNGIAMVDYLQKAVLLNLGTIELYGSNDPYRREPRSPRKSRQPSGAGLCDISEGTVVPEDRCKSPTSGSSSPHNSDDEQKMNNFIEKVKTKSRKFSKMVANDIAKMSRKLSLPTDLKPDLDVKDNSFSRSRSSSVTSIDKESREAISALHFCETFTRKTDSSPSPCLWVGTTLGTVLVIALNLPPGGEQRLLQPVIVSPSGTILRLKGAILRMAFLDTTGCLIPPAYEPWREHNVPEEKDEKEKLKKRRPVSVSPSSSQEISENQYAVICSEKQAKVISLPTQNCAYKQNITETSFVLRGDIVALSNSICLACFCANGHIMTFSLPSLRPLLDVYYLPLTNMRIARTFCFTNNGQALYLVSPTEIQRLTYSQETCENLQEMLGELFTPVETPEAPNRGFFKGLFGGGAQSLDREELFGESSSGKASRSLAQHIPGPGGIEGVKGAASGVVGELARARLALDERGQKLGDLEERTAAMLSSAESFSKHAHEIMLKYKDKKWYQF

SEQ ID NO: 5 - DNA, 3348 bp, Homo sapiens
  CDS (1)..(3348)
atgaggaaattcaacatcaggaaggtgctggacggcctgaccgccggctcgtcctcggcgtcgcagcagcaacagcagcagcatccgcctgggaaccgggagccggagatccaggaaacgctccagtccgagcactttcagctctgcaagactgttcgccatggatttccctatcaaccctcagccctggcctttgatcctgtacagaagatcctggcagtgggaactcagactggtgctttaaggctctttggtcgtccaggagtagaatgttattgccagcatgacagtggagctgcagtaatccagctccagttcctgattaatgagggagcgcttgtgagtgccttggctgatgacaccttacacttatggaatttacgtcagaagaggcctgccatactacattcgcttaaattttgcagagaaagggttacattttgccatctgcctttccagagtaagtggctctatgtgggcactgaacgaggtaatatacatattgtcaatgtggagtccttcacactctcaggctacgtcattatgtggaataaagccattgaactgtcatctaaatctcacccaggacctgtggtccatataagtgataatccaatggacgagggaaagcttttgattggctttgaatctggaacagtagttttatgggacctcaaatcaaagaaagccgactacagatacacatatgatgaggctatccactctgttgcttggcatcatgaaggaaaacaatttatttgcagtcattcagatggcaccttgactatatggaatgtaaggtcccctgctaaaccagtacagacaatcactccacatggaaaacagttaaaggatgggaagaagccagaaccatgcaaacctatcctcaaggtggaattcaaaacgactagatctggggagccttttattattttatcaggaggtttgtcatatgatactgtaggaagaagaccttgcttaacagtgatgcatgggaaaagcactgctgtgctagaaatggactattcaattgttgattttctaacgctgtgtgaaacaccatacccaaatgattttcaagaaccatatgctgtggttgttcttctagaaaaggatttagtacttatagaccttgcacaaaatggatatcctatatttgaaaatccctaccctttgagtatacatgagtcccctgttacatgttgcgaatattttgcggattgtcctgtggaccttattcctgcactttattctgttggagctagacagaaacgtcaaggttacagcaaaaaggaatggcccatcaacggaggtaattggggcttgggtgctcaaagttacccagaaataattattacagggcatgctgatgggtcagttaagttctgggatgcttctgcaataactctacaagtattatataagctaaagacatctaaagtatttgaaaagtcaagaaataaagatgacaggccaaacacagacattgtagatgaagatccatatgccattcagatcatctcctggtgtccagaaagtagaatgctgtgcatcgctggagtttcagctcatgtcattatttatagattcagcaagcaggaagtaatcacagaagtcattccgatgcttgaagttcgattattatatgagataaatgatgtggaaactccggagggtgagcagccaccacctttgccaacacccgtgggagggtccaaccctcagcccatccctcctcagtctcatccatctaccagtagcagttcatctgatgggcttcgtgataatgtaccttgtttaaaagttaaaaactcaccacttaaacagtctccaggttatcaaacagaactagttattcagttggtttgggtgggtggagaaccaccacaacaaataaccagcctggcagtcaattcttcctatggactggtggtttttggcaattgcaatggcattgctatggttgactacctccagaaagcagtgctgctcaacctgggcactattgaattatatggctctaatgatccttatcggagagaaccccgatctcctcgtaaatctcgacagccttcaggagccggtctgtgtgatattagtgaagggactgttgttccagaggatcgctgcaaatctccaacctctgcaaagatgtcaaggaagttaagcttacctactgacctaaagcctgatttagatgtaaaggataactcctttagccgatcacggagttcaagtgtaacaagcattgacaaagaatcccgagaagcgatctccgctcttcatttctgtgaaacgtttactcgaaagacggactcgtccccttccccttgtctgtgggttggaacaacgctaggaacagtgcttgtcattgcactgaaccttcccccagggggagagcaaagacttcttcagccagtaattgtgtctccaagtggtactatattgaggttaaaaggtgcaatcttgagaatggcatttctggataccacaggctgcttaataccacctgcgtatgaaccctggagagagcacaatgttcctgaagaaaaagacgaaaaggagaaattgaaaaaacggcggcctgtctcagtatccccctcctcttctcaggaaattagtgaaaaccagtatgcagtgatatgttctgaaaagcaagcaaaagtaatctcactgccaacccagaactgtgcttataagcaaaatattacagagacctcgtttgtgcttcgtggagatattgtagcattgagtaacagtatctgccttgcctgtttctgtgccaatggacatataatgacttttagtttgccaagtttaagacctctgttggatgtgtattacttgccccttaccaatatgcggatagccagaacgttctgctttaccaacaatggacaagcattataccttgtttcacctacagaaatccagagacttacttatagtcaagagacctgtgaaaatcttcaggaaatgttgggtgaactcttcactcctgtagaaacacctgaagcaccaaacaggggattctttaaaggcttatttggaggtggtgcacaatctcttgacagagaagaactatttggagaatcgtcctcaggaaaggcttcaaggagccttgcacagcatattcctggccctggtggcattgaaggcgtaaaaggggcagcatctggagttgttggtgaattagcacgagccaggctggcactagatgaaagagggcagaaacttggcgatctggaagaaagaactgcggccatgttatcaagtgcagagtcattttctaaacatgctcatgagattatgttgaaatacaaagataagaagtggtaccagttctga

SEQ ID NO: 6 - protein, 1115 aa, Homo sapiens
MRKFNIRKVLDGLTAGSSSASQQQQQQHPPGNREPEIQETLQSEHFQLCKTVRHGFPYQPSALAFDPVQKILAVGTQTGALRLFGRPGVECYCQHDSGAAVIQLQFLINEGALVSALADDTLHLWNLRQKRPAILHSLKFCRERVTFCHLPFQSKWLYVGTERGNIHIVNVESFTLSGYVIMWNKAIELSSKSHPGPVVHISDNPMDEGKLLIGFESGTVVLWDLKSKKADYRYTYDEAIHSVAWHHEGKQFICSHSDGTLTIWNVRSPAKPVQTITPHGKQLKDGKKPEPCKPILKVEFKTTRSGEPFIILSGGLSYDTVGRRPCLTVMHGKSTAVLEMDYSIVDFLTLCETPYPNDFQEPYAVVVLLEKDLVLIDLAQNGYPIFENPYPLSIHESPVTCCEYFADCPVDLIPALYSVGARQKRQGYSKKEWPINGGNWGLGAQSYPEIIITGHADGSVKFWDASAITLQVLYKLKTSKVFEKSRNKDDRPNTDIVDEDPYAIQIISWCPESRMLCIAGVSAHVIIYRFSKQEVITEVIPMLEVRLLYEINDVETPEGEQPPPLPTPVGGSNPQPIPPQSHPSTSSSSSDGLRDNVPCLKVKNSPLKQSPGYQTELVIQLVWVGGEPPQQITSLAVNSSYGLVVFGNCNGIAMVDYLQKAVLLNLGTIELYGSNDPYRREPRSPRKSRQPSGAGLCDISEGTVVPEDRCKSPTSAKMSRKLSLPTDLKPDLDVKDNSFSRSRSSSVTSIDKESREAISALHFCETFTRKTDSSPSPCLWVGTTLGTVLVIALNLPPGGEQRLLQPVIVSPSGTILRLKGAILRMAFLDTTGCLIPPAYEPWREHNVPEEKDEKEKLKKRRPVSVSPSSSQEISENQYAVICSEKQAKVISLPTQNCAYKQNITETSFVLRGDIVALSNSICLACFCANGHIMTFSLPSLRPLLDVYYLPLTNMRIARTFCFTNNGQALYLVSPTEIQRLTYSQETCENLQEMLGELFTPVETPEAPNRGFFKGLFGGGAQSLDREELFGESSSGKASRSLAQHIPGPGGIEGVKGAASGVVGELARARLALDERGQKLGDLEERTAAMLSSAESFSKHAHEIMLKYKDKKWYQF

SEQ ID NO: 7 - DNA, 5184 bp, Homo sapiens
  CDS (317)..(4096)
gacagcacagagctcaggaacgctgcctgaggaccctggggcctacgaggaggagaagagggcaggagcgcttgcagagaccctgggctcctatcctgccataagcctcgctgtctcctgatatctgcagccaggccctactgacacccccaggcctgagtgcaagcagagaccccaccattcccaggccctggaggactggtccaccttaactgggcagcccttggggcaggcgctggccggtgcctcagcccaggcctctgtgctctgcatgcactgccagcctgccatcaggcctctattgcagccctgaaccatgatccagggcaccttggagccagatggtcccctctggggctgggactgggacagtgacaatgactgggatagtgctgtgctggccctcctggcgctggctgtggtggctgccacagcgctggccttacactggtttggctccgggcacgatcaagaggcggcagaaccggtgtccacagccctcggggctcaacctcatcaggcaggaggagctgagctggccctgcaaccgaagtctaaggtcagtgatggcagcgaggggcagagcccagggcaggggaaaccagagcccccaggacgcggccagcagagccctgtccctgctgcagcgccgggcgggggcctggccgccatggcccggcttccactcaagacggctgtcgaggaggcccgcagagaggcattaggacagcaacggggcagtgccacccccgcggccccccgagcggaaggaaaggagcctcccaggccaggcactgccctcctgggcaggagcgaagcaggggggatgtccgcccccctcctgatccacttcactcctcggagccctggcagcgaagcggaggcggagacaggtggtgtcagggcgtcctctcgccaggccgcaggccccgcggggcaacaggacactggcccctggcaggcgggcgcggggccctcgggctcgatggggagaggccggggccggcggcggcggatggacgctggctcgggagacagagcccgccgcccccggaaactggacccgctccgcctgggcgccgcggggagcgtgtgggacgcggtggacggggccgccgccctggacgcccacgcgcgcggcctccccacaggacccccactcgcccaggagcccgcactcccggcgctgcccgctccccgcgccctgcagcctgggtctcagacggaaggctctggggccaagggtggctggagcagggaggcctcgggggtccctgcccccggaggaggctggccctgggtcagcagggaggtcccgggcacccggagctttggcccagccccaggctccacgcgcccctggctagagagtccgcctcaaggtcgcccactctcgtcccaagggccgggtgccacaggggcctacgatgccggcgaggccggggctgacagctcccgagataacagtcctgccgctgacctggggcccacccggcccccggagcaagcaaagccggccgcagccggccacagccgcgcgccctcccggagccgtgagcctcgcccgcgctccgcctccccgcccgcagctcccggcccggggttcccacctgaagccctgactctcccctctccttcagactttttgcccctggaggttacccaggatccttccgtgggcgaaaatctcagagcggcgccagccccaagttcagcctcagcccaagtcttaacttcagctccagcctcagtcctagccccagccctggcttcatcccccagctcagcaccaacctcagccaccacctcaacctcatcccccacctcagccccagccccagctccaacctcagctccaacttcaaccccagccccagccccaagtccagctgcagccgcaactccagccccagccccagtcccagtcccaaccctcacacccccatccccagccctaaccccagtcccaaccccagccctaagcccagctccaactccagccccaaccccagccgcatccccagccctaaccccagtcccaaccccagccctaagcccagctccaactccagccctaaccccagccgcatccccagccctaaccccagtcccaaccccagccctaagcccagctccaactccagccccaaccccagccgcatcccctgccccagcccccacctcagccccaaccccaaccccagccgcatcccctgccccagctgacgggtcaaagcctcaggagagtgtggctctccccaggcgctaccaggaggggcaggtctcagccagctggggaaaccttattgccatggttcttagaagccaccccttccccaggcaagacaggccccaagggagtgtcccgagggcggttcccgggagccccgtgggtcccagcacttccacacactctgaggacagacacggcccctcttcttcagtggggacagtcatagggacaggtacagggggcctggttgaggctggaggtcagccacagccaagaagctccgagaccaacggatcgcccagcccagaccctcccccaggcctaagaggagagggaaccagggagaaaagtctagacccgctgccccaagccgcgatgcccaggggccccgcacagccccccgcgcagaggccgcctggccccgcggcctcctcctctgcgaggcgctcacagccggtaccccagctacggaaacgcagcaggtgcgaaatcgccccgagctcggagcaggaggtcaggccggccgcctcgggggaccctcaaggggaggcgccgggggaggggggcagccctgccggccgcagctgggcgctcacggaaaagcaggaggaggcccggaagctcatggtgtttctgcagaggcccgggggttgggggttggtggaggggccccggaagcccagctcccgggccctggagcccgccacggcggcagccctgcggcggcggctggacctgggcagttgcctggacgtgctggcctttgcccggcagcacggagagcccggcctggcgcaggagacctacgcgctgatgagcgacaacctgctgcgagtgctgggagacccgtgcctctaccgccggctgagcgcggccgaccgcgagcgcatcctcagcctgcggaccggccggggccgggcggtgctgggcgtcctcgtactgcccagcctctaccaggggggccgctcagggctccccaggggccctcgtggcgaggagcctcctgcggcggcccctgtgtccctgcctctacctgcgcacctgcatgtgttcaacccccgggagaacacctggcggcccctgacccaggtgcccgaggaggccccgcttcggggctgcggtctctgcaccatgcacaactacctgtttctggcggggggcatccgtggctccggtgccaaggccgtctgctccaacgaggtcttctgctacaaccctctgaccaacatctggagccaggttcggcccatgcagcaggcccgagcccagctcaagctggtggccctggacgggctgctctatgccatcggtggcgaatgcctgtacagcatggagtgctacgacccgcgaacagacgcctggaccccacgcgcgccactccccgcaggcaccttccctgtggcccacgaggctgtggcctgccgtggggacatctacgtcaccgggggtcacctcttctaccgcctgctcaggtacagccccgtgaaggatgcttgggacgagtgcccatacagtgccagccaccggcgttccagcgacatcgtggcactggggggcttcctgtaccgcttcgacctgctgcggggcgtgggcgccgccgtgatgcgctacaacacagtgaccggctcctggagcagggctgcctccctgcccctgcccgcccccgccccactgcgctgcaccaccctgggcaacaccatttactgcctcaacccccaggtcactgccaccttcacggtctctggggggactgcccagttccaggccaaggagctgcagcccttccccttggggagcaccggggtcctcagtccattcatcctgactctgccccctgaggaccggctgcagacctcactctgagtggcaggcagagaaccaaagctgcttcgctgctctccagggagaccctcctgggatgggcctgagaggccgggactcagggaaggggctgggatcggaacttcctgctcttgtttctggacaactttccccttctgctttaaaggttgtcgattattttgaagcccagactccctcagcctctttctgcccctcactccacacccagactgtttcctgactcaattccgtacctacttacagaccctctcagcttgctgacacccccctgtctgtgggactccctattccctagagccagggactgatgtgtccccacagacaaggacttggctcgctggagctctgctgagccgagagaggagggggtagaaaacattcacacttcctatgctctgtcagcaggacagggagcaaaaacgtccccaggcaacgccctcgcctctgtgactttctgcctgtcctaaggcctccccaggtaccaaccccgtagctatctgggtctgtttggcactgtggattctcaagggcctagaacccttgcctctgaaactggtccgctggtgcagccctgctgtctgcagctcctgcccatacccccagcccacaccaggccaggcccactccgggctcaccaccctctgcagccttgtggggctctcccagcccctccagaagcccaccccacttctcgccaacccccgatctctaaatgaggcctgagcgtcaccctagttctgcccctttttagctgtgtagacttggacgagacatttgacttccctttctccttgtctataaaatgtggacagtggacgtctgtcacccaagagagttgtgggagacaagatcacagctatgagcacctcgcacggtgtccaggatgcacagcacaatccatgatgtgttttctccccttacgcactttgaaacccatgctagaaaggtgaatacatctgactgtgctccactccaacctccagcctggatgtccctgtctgggccctttttctgttttttattctatgttcagcaccactggcaccaaatacattttaattcaccgaaagcaaaaaaaaaaaaaaaaa

SEQ ID NO: 8 - protein, 1259 aa, Homo sapiens [sequence omitted]

SEQ ID NO: 9 - DNA, 1053 bp, Homo sapiens
  CDS (1)..(1053)
atgctcgaaggtgcggagctgtacttcaacgtggaccatggctacctggagggcctggttcgaggatgcaaggccagcctcctgacccagcaagactatatcaacctggtccagtgtgagaccctagaagacctgaaaattcatctccagactactgattatggtaactttttggctaatcacacaaatcctcttactgtttccaaaattgacgctgagatgaggaagagactatgtggagaatttgagtatttccggaatcattccctggagcccctcagcacatttctcacctatatgacgtgcagttatatgatagacaatgtgattctgctgatgaatggtgcattgcagaaaaaatctgtgaaagaaattctggggaagtgccaccccttgggccgtttcacagaaatggaagctgtcaacattgcagagacaccttcagatctctttaatgccattctgatcgaaacgccattagctccattcttccaagactgcatgtctgaaaatgctctagatgaactgaatattgaattgctacgcaataaactatacaagtcttaccttgaggcattctataaattctgtaagaatcatggtgatgtcacagcagaagttatgtgtcccattcttgagtttgaggccgacagacgtgcttttatcatcactcttaactcctttggcactgaattgagcaaagaagaccgagagaccctctatccaaccttcggcaaactctatcctgaggggttgcggctgttggctcaagcagaagactttgaccagatgaagaacgtagcggatcattacggagtatacaaacctttatttgaagctrtaggtggcagtgggggaaagacattggaggacgtgttttacgagcgtgaggtacaaatgaatgtgctggcattcaacagacagttccactacggtgtgttttatgcatatgtaaagctgaaggaacaggaaattagaaatattgtgtggatagcagaatgtatttcacagaggcatcgaactaaaatcaacagttacattccaattttataa

SEQ ID NO: 10 - protein, 350 aa, Homo sapiens
  misc_feature (281)..(281): The ′Xaa′ at location 281 stands for Val, or Ile.
MLEGAELYFNVDHGYLEGLVRGCKASLLTQQDYINLVQCETLEDLKIHLQTTDYGNFLANHTNPLTVSKIDAEMRKRLCGEFEYFRNHSLEPLSTFLTYMTCSYMIDNVILLMNGALQKKSVKEILGKCHPLGRFTEMEAVNIAETPSDLFNAILIETPLAPFFQDCMSENALDELNIELLRNKLYKSYLEAFYKFCKNHGDVTAEVMCPILEFEADRRAFIITLNSFGTELSKEDRETLYPTFGKLYPEGLRLLAQAEDFDQMKNVADHYGVYKPLFEAXGGSGGKTLEDVFYEREVQMNVLAFNRQFHYGVFYAYVKLKEQEIRNIVWIAECISQRHRTKINSYIPIL

SEQ ID NO: 11 - DNA, 1197 bp, Homo sapiens
  CDS (1)..(1194)
atgaggaagcggaccgagcccgtcgccttggagcatgagcgctgcgccgccgcgggctcgtcctcctccggctcggccgccgcggcgctggacgccgactgccgcctgaagcagaacctacgcctgacgggcccggcggcggctgagccgcgctgcgcagccgacgcgggaatgaagcgggcgctgggcaggcgaaagggcgtgtggttgcgcctgaggaagatacttttctgtgttttggggttgtacattgccattccatttctcatcaaactatgtcctggaatacaggccaaactgattttcttgaatttcgtaagagttccctatttcattgatttgaaaaaaccacaggatcaaggtttgaatcacacgtgtaactactacctgcagccagaggaagacgtgaccattggagtctggcacaccgtccctgcagtctggtggaagaacgcccaaggcaaagaccagatgtggtatgaggatgccttggcttccagccaccctatcattctgtacctgcatgggaacgcaggtaccagaggaggcgaccaccgcgtggagctttacaaggtgctgagttcccttggttaccatgtggtcacctttgactacagaggttggggtgactcagtgggaacgccatctgagcggggcatgacctatgacgcactccacgtttttgactggatcaaagcaagaagtggtgacaaccccgtgtacatctggggccactctctgggcactggcgtggcgacaaatctggtgcggcgcctctgtgagcgagagacgcctccagatgcccttatattggaatctccattcactaatatccgcgaagaagctaagagccatccattttcagtgatatatcgatacttccctgggtttgactggttcttccttgatcctattacaagtagtggaattaaatttgcaaatgatgaaaacgtgaagcacatctcctgtcccctgctcatcctgcacgctgaggacgacccggtggtgcccttccagcttggcagaaagctctatagcatcgccgcaccagctcgaagcttccgagatttcaaagttcagtttgtgccctttcattcagaccttggctacaggcacaaatacatttacaagagccctgagctgccacggatactgagggaattcctggggaagtcggagcctgagcaccagcactga

SEQ ID NO: 12 - protein, 398 aa, Homo sapiens
MRKRTEPVALEHERCAAAGSSSSGSAAAALDADCRLKQNLRLTGPAAAEPRCAADAGMKRALGRRKGVWLRLRKILFCVLGLYIAIPFLIKLCPGIQAKLIFLNFVRVPYFIDLKKPQDQGLNHTCNYYLQPEEDVTIGVWHTVPAVWWKNAQGKDQMWYEDALASSHPIILYLHGNAGTRGGDHRVELYKVLSSLGYHVVTFDYRGWGDSVGTPSERGMTYDALHVFDWIKARSGDNPVYIWGHSLGTGVATNLVRRLCERETPPDALILESPFTNIREEAKSHPFSVIYRYFPGFDWFFLDPITSSGIKFANDENVKHISCPLLILHAEDDPVVPFQLGRKLYSIAAPARSFRDFKVQFVPFHSDLGYRHKYIYKSPELPRILREFLGKSEPEHQH

SEQ ID NO: 13 - DNA, 324 bp, Homo sapiens
  CDS (1)..(321): First open reading frame
atggcactgcctcaggcccagctccacctcctccctgcaccctcccagcagttgctgcagatgagagcggcgatgggaccagggtccagagcaatggaggctccgtgcctgggagcgggttctgccggctgcggaaggataggggtggcacagtcagccgccttggggacacaggacataggggacccaccaccgccactgccgctcccgcagcagctcgtgccgccaccacccacgcctccttgctgcagccagcgtgacgacagcggctgctccggaccacccgcaagcggccatcacggcaagggactaaccgtcgtgtaa

SEQ ID NO: 14 - protein, 107 aa, Homo sapiens
MALPQAQLHLLPAPSQQLLQMRAAMGPGSRAMEAPCLGAGSAGCGRIGVAQSAALGTQDIGDPPPPLPLPQQLVPPPPTPPCCSQRDDSGCSGPPASGHHGKGLTVV

SEQ ID NO: 15 - DNA, 260 bp, Homo sapiens
  CDS (9)..(257): Second open reading frame
gagcggcgatgggaccagggtccagagcaatggaggctccgtgcctgggagcgggttctgccggctgcggaaggataggggtggcacagtcagccgccttggggacacaggacataggggacccaccaccgccactgccgctcccgcagcagctcgtgccgccaccacccacgcctccttgctgcagccagcgtgacgacagcggctgctccggaccacccgcaagcggccatcacggcaagggactaaccgtcgtgtaa

SEQ ID NO: 16 - protein, 83 aa, Homo sapiens
MGPGSRAMEAPCLGAGSAGCGRIGVAQSAALGTQDIGDPPPPLPLPQQLVPPPPTPPCCSQRDDSGCSGPPASGHHGKGLTVV